(19) United States
(12) Patent Application Publication
(10) Pub. No.: US 2009/0093873 A1
(43) Pub. Date: Apr. 9, 2009

(54) VASCULAR GRAFT AND METHOD OF USE

(75) Inventor: Jose L. Navia, Shaker Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/238,208

(22) Filed: Sep. 25, 2008

Related U.S. Application Data
(60) Provisional application No. 60/976,022, filed on Sep. 28, 2007.

(51) Int. Cl. A61F 2/06

(52) U.S. Cl. 623/1.23; 623/1.36; 623/1.35

(57) ABSTRACT

A vascular graft includes an elongated main body portion, a first sewing ring, a second sewing ring, and a third sewing ring. The elongated main body portion includes a first end defining a first opening, a second end defining a second opening, and an aperture defined by a perimeter. The first sewing ring is securely attached to the perimeter of the aperture. The second sewing ring is securely attached to the first end of the main body portion adjacent the first opening. The third sewing ring is securely attached to the second end of the main body portion adjacent the second opening.

### RELATED APPLICATION

This application claims priority from U.S. provisional patent application Ser. No. 60/976,022, filed Sep. 28, 2007, the entirety of which is hereby incorporated by reference.

### TECHNICAL FIELD

The present invention relates generally to vascular repair of bodily vessels, and more particularly to a vascular graft and related method for repairing vessel abnormalities.

### BACKGROUND OF THE INVENTION

A leading cause of disability and death in both the U.S. and abroad includes damage to a portion of the vascular system. This is a particular problem with regard to aortic aneurysms. Diseases of the aorta, for example, are becoming an increasing concern as a result of advancements in cardiac surgery and human longevity. Severe arterial sclerosis, severely calcified aorta, and other indications continue to necessitate complete or partial aortic replacement procedures.

Aneurysms are typically characterized by diseased or damaged blood vessels which lead to a weakening of the vessel wall. Weakening of the vessel wall can then lead to a blood-filled dilation of the vessel. Left untreated, aneurysms will eventually rupture and result in acute (and often fatal) hemorrhaging in a very short period of time.

The aorta has numerous arterial branches. The arch of the thoracic aorta, for example, has three major branches arising from the convex upper surface of the arch and ascending through the superior thoracic aperture to the root of the neck. The proximity of an aneurysm to a branch artery may limit the use of an excluding device, such as a tubular stent graft. For example, the main body or ends of a tubular stent graft may occlude or block the branch arteries as a result of positioning the stent graft against a healthy, i.e., non-diseased or dilated portion of the artery wall. Additionally, there may be an inadequate length of healthy tissue for the stent graft to seal against in the area between the aneurysm and the location of the branch arteries. Even if the stent graft is initially located without blocking a branch artery, there still is a risk, that the devices will migrate to a position where it may partially or fully block a branch artery.

### SUMMARY OF THE INVENTION

In one aspect of the present invention, a vascular graft comprises an elongated main body portion, a first sewing ring, a second sewing ring, and a third sewing ring. The elongated main body portion includes a first end defining a first opening, a second end defining a second opening, and an aperture defined by a perimeter. The first sewing ring is securely attached to the perimeter of the aperture. The second sewing ring is securely attached to the first end of the main body portion adjacent the first opening. The third sewing ring is securely attached to the second end of the main body portion adjacent the second opening.

In another aspect of the present invention, a vascular graft comprises a Y-shaped tubular main body portion having first and second ends. The first end defines a first opening and has a first sewing ring securely attached thereto. The second end includes first and second leg members fluidly connected with the main body portion. Each of the first and second leg members includes an end defined by an opening and has second and third sewing rings respectively attached thereto. The main body portion includes a fourth sewing ring securely attached thereto.

In another aspect of the present invention, a method is provided for repairing at least a portion of a blood vessel. One step of the method includes providing a vascular graft at least partially encapsulated within a delivery sheath. The vascular graft includes an elongated main body portion, a first sewing ring, a second sewing ring, and a third sewing ring. The first sewing ring is securely attached to an aperture of the main body portion. The second sewing ring is securely attached to a first end of the main body portion adjacent a first opening. The third sewing ring is securely attached to a second end of the main body portion adjacent a second opening. The delivery sheath comprises first and second envelope members. Each of the first and second envelope members includes a release mechanism. Next, a placement position is determined for the vascular graft at the portion of the blood vessel to be repaired. The vascular graft is then delivered to the portion of the blood vessel to be repaired, whereafter the delivery sheath is manipulated so that the vascular graft expands into the portion of the blood vessel to be repaired. The vascular graft is then secured to the portion of the blood vessel to be repaired.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1A is a perspective view showing a vascular graft having an exploded configuration and constructed in accordance with the present invention;

FIG. 1B is a perspective view of the vascular graft of FIG. 1A in an assembled configuration;

FIG. 1C is a perspective view of an expandable support member;

FIG. 1D is a perspective view showing an alternative embodiment of the expandable support member in FIG. 1C;

FIG. 1E is a perspective view showing the vascular graft of FIG. 1A including the expandable support member of FIG. 1C;

FIG. 2A is a perspective view showing an alternative embodiment of the vascular graft in FIG. 1A;

FIG. 2B is a perspective view showing an alternative embodiment of the vascular graft in FIG. 2A;

FIG. 3A is a perspective view showing an alternative embodiment of the vascular graft of FIG. 1A in an exploded configuration;

FIG. 3B is a perspective view of the vascular graft of FIG. 3A in an assembled configuration;

FIG. 3C is a perspective view showing an alternative embodiment of the vascular graft in FIGS. 3A and 3B;

FIG. 4A is a perspective view showing another alternative embodiment of the vascular graft of FIG. 1A in an exploded configuration;

FIG. 4B is a perspective view of the vascular graft of FIG. 4A in an assembled configuration;

FIG. 5A is a perspective view showing an alternative embodiment of the vascular graft of FIG. 3A in an exploded configuration;

FIG. 5B is a perspective view of the vascular graft of FIG. 5A in an assembled configuration;

FIG. 5C is a perspective view showing an alternative embodiment of the vascular graft in FIGS. 5A and 5B;

FIG. 6A is a perspective view showing an alternative embodiment of the vascular graft in FIG. 4A;

FIG. 6B is a perspective view showing an alternative embodiment of the vascular graft in FIG. 6A;

FIG. 7A is a perspective view showing an alternative embodiment of the vascular graft in FIG. 5A;

FIG. 7B is a perspective view showing an alternative embodiment of the vascular graft in FIG. 7A;

FIG. 8A is a perspective view of an alternative embodiment of a Y-shaped vascular graft for implantation in the iliac arteries;

FIG. 8B is a perspective view showing an alternative embodiment of the vascular graft in FIG. 8A;

FIG. 9A is a perspective view of another alternative embodiment of a vascular graft for implantation in a descending abdominal aorta;

FIG. 9B is a perspective view showing an alternative embodiment of the vascular graft in FIG. 9A;

FIG. 10 is a perspective view of an abdominal aorta having a diseased portion (shaded region);

FIG. 11 is a perspective view of the abdominal aorta in FIG. 10 with the diseased portion resected;

FIG. 12A is a perspective view showing the vascular graft of FIG. 4B being delivered to the abdominal aorta in a delivery sheath;

FIG. 12B is a perspective view showing the vascular graft of FIG. 12A upon removal from the delivery sheath;

FIG. 12C is a perspective view showing a first end of the vascular graft in FIG. 12B anastomosed with a portion of the abdominal aorta;

FIG. 12D is a perspective view showing a second end of the vascular graft in FIG. 12C anastomosed with a portion of the abdominal aorta;

FIG. 13 is a perspective view showing the vascular graft of FIG. 12D implanted in the abdominal aorta;

FIG. 14 is a perspective view showing the vascular graft of FIG. 3B being delivered to an aortic arch aneurysm;

FIG. 15 is a perspective view showing a second main body portion of the vascular graft in FIG. 14 expanded in the proximal descending aorta;

FIG. 16 is a perspective view showing a main body portion of the vascular graft in FIG. 15 expanded in the aortic arch;

FIG. 17 is a perspective view showing the vascular graft of FIG. 3B implanted in the aortic arch aneurysm:

FIG. 18 is a perspective view showing a second main body portion of the vascular graft in FIG. 5B being delivered to an aortic arch aneurysm;

FIG. 19 is a perspective view showing the main body portion of the vascular graft in FIG. 5B anastomosed with the second main body portion in FIG. 18;

FIG. 20 is a perspective view showing the main body portion of the vascular graft in FIG. 5B expanded in the aortic arch;

FIG. 21 is a perspective view of the vascular graft in FIG. 5B implanted in the aortic arch aneurysm;

FIG. 22 is a perspective view of an abdominal aorta having a diseased portion (shaded region);

FIG. 23 is a perspective view of the abdominal aorta in FIG. 22 with the diseased portion resected;

FIG. 24A is a perspective view showing the vascular graft of FIG. 18B being delivered to the abdominal aorta in a delivery sheath;

FIG. 24B is a perspective view showing the vascular graft of FIG. 24A upon removal from the delivery sheath;

FIG. 24C is a perspective view showing a first end of the vascular graft in FIG. 24B anastomosed with a portion of the abdominal aorta;

FIG. 24D is a perspective view showing a second end of the vascular graft in FIG. 24C anastomosed with a portion of the abdominal aorta;

FIG. 25 is a perspective view showing the vascular graft of FIG. 24D implanted in the abdominal aorta;

FIG. 26 is a perspective view showing a second main body portion of the vascular graft in FIG. 19A being delivered to an aortic arch aneurysm;

FIG. 27 is a perspective view showing the main body portion of the vascular graft in FIG. 19A anastomosed with the second main body portion in FIG. 26;

FIG. 28 is a perspective view of the vascular graft in FIG. 19A implanted in the aortic arch aneurysm;

FIG. 29 is a perspective view showing the vascular graft of FIG. 3B being delivered to the abdominal aorta in a delivery sheath;

FIG. 30 is a perspective view showing a portion of the vascular graft in FIG. 29 upon removal from the delivery sheath;

FIG. 31 is a perspective view showing the vascular graft in FIG. 30 implanted in the abdominal aorta;

FIG. 32 is a perspective view showing the vascular graft of FIG. 2B being delivered to an aortic arch aneurysm;

FIG. 33 is a perspective view showing a second main body portion of the vascular graft in FIG. 32 expanded in the proximal descending aorta;

FIG. 34 is a perspective view showing a main body portion of the vascular graft in FIG. 33 expanded in the aortic arch: and

FIG. 35 is a perspective view showing the vascular graft of FIG. 34 implanted in the aortic arch aneurysm.

### DETAILED DESCRIPTION

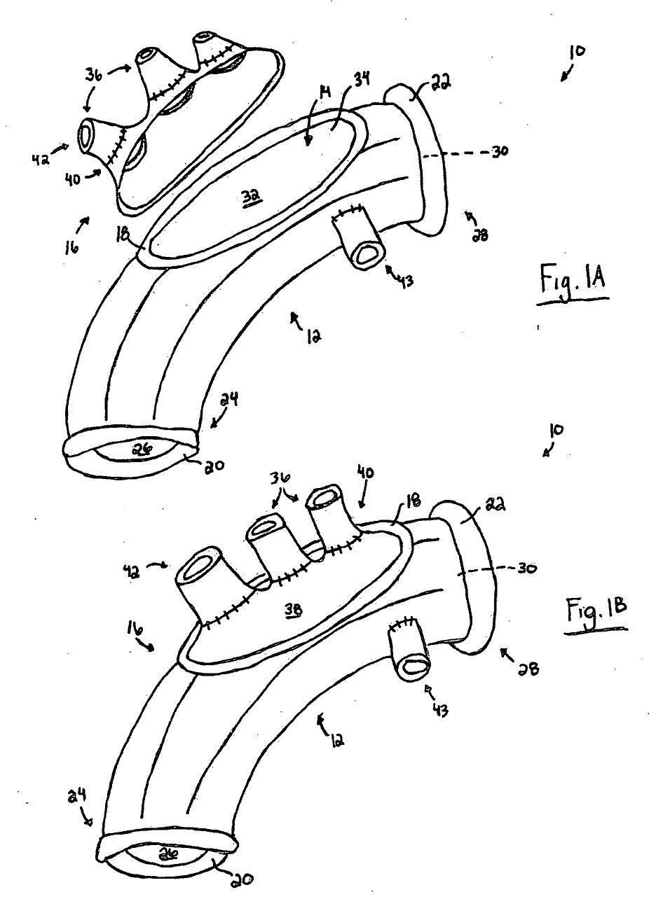

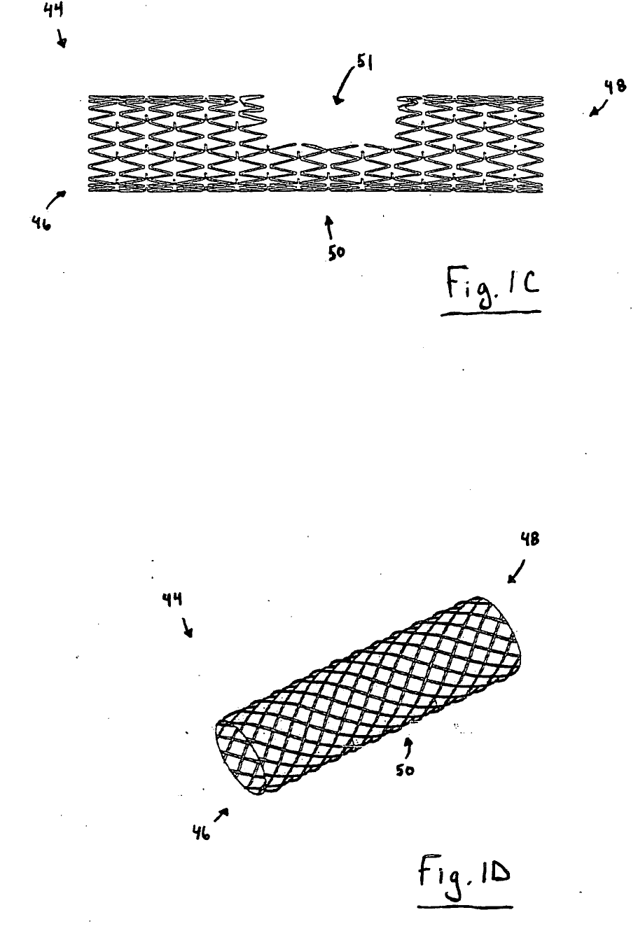

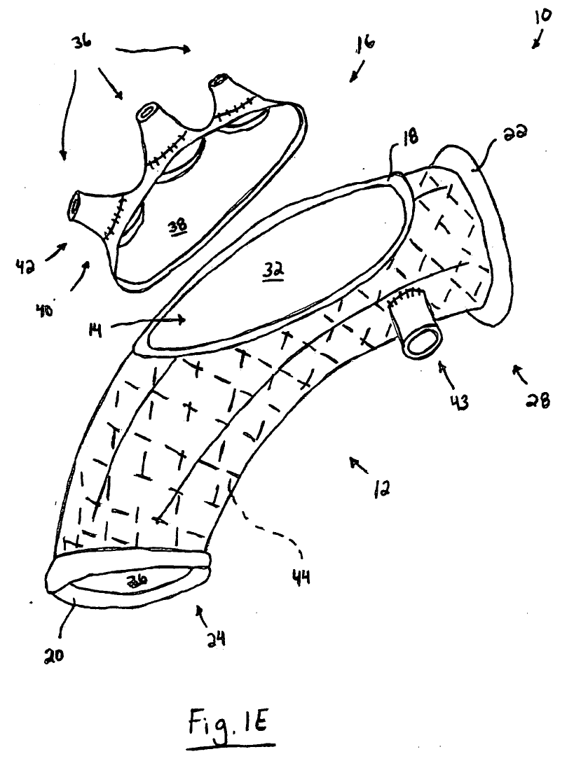

The present invention relates generally to vascular repair of bodily vessels, and more particularly to a vascular graft and related method for repairing vessel abnormalities. As representative of the present invention, FIGS. 1A and 1B illustrate a vascular graft 10 for the treatment of vascular abnormalities, such as aortic and/or abdominal aneurysms. The vascular graft 10 can be utilized in the thoracic aorta, for example, and can be used to repair thoracic aneurysms or thoracic dissecting aneurysms. Further, the vascular graft 10 may also be used to treat vascular trauma, arteriosclerosis, calcification, microbial infection, congenital defects, and other obstructive diseases associated with the aorta. Accordingly, the term “aortic aneurysm” as used herein is intended to relate to and include thoracic aneurysms, abdominal aneurysms, and related vessel diseases.

FIGS. 1A and 1B illustrate one embodiment of the present invention. In FIGS. 1A and 1B, a vascular graft 10 comprises an elongated main body portion 12 having an aperture 14 for anastomosis with a multi-lumen branch graft 16, a first sewing ring 18, a second sewing ring 20, and a third sewing ring 22. The vascular graft 10 has a generally flexible, tube-like configuration and is adapted for placement in a bodily vessel, such as an artery or vein. The vascular graft 10 is configured to engage an inner surface of a bodily vessel so that the main body portion 12 forms a substantial seal with the inner surface of the bodily vessel. As described in more detail below, the vascular graft 10 can be compressed to facilitate delivery to a bodily vessel and then selectively expanded by, for example, a balloon, stent, etc. so that the vascular graft substantially conforms to the inner surface of the bodily vessel.

The material comprising the vascular graft 10 can include any biocompatible material that is mechanically stable in vivo and is capable of preventing or substantially reducing the possibility of the passage or flow of blood or other body fluids through the vascular graft. Examples of suitable materials for use in constructing the vascular graft 10 can include biocompatible plastics, such as woven polyester, non-resorbable elastomers or polymers such as silicone, SBR, EPDM, butyl, polyisoprene, Nitril, Neoprene, nylon alloys and blends, poly(ethylene-vinyl-acetate) (EVA) copolymers, silicone rubber, polyamides, polyurethane, poly(ester urethanes), poly(ether urethanes), poly(ester urea), polypropylene, polyethylene, polycarbonate, polytetrafluoroethylene (PTFE) (e.g., TEFLON), expanded PTFE (ePTFE), polyethylene terephthalate (e.g., DACRON), and polyethylene copolymers.

The vascular graft 10 can also include a layer of biological material (not shown), such as bovine or equine pericardium, peritoneal tissue, an allograft, a homograft, a patient graft, or a cell-seeded tissue. The layer can cover the entire vascular graft 10 or only a portion thereof. One skilled in the art will appreciate that other materials suitable for vascular surgical applications may also be appropriate for the vascular graft 10.

Referring again to FIGS. 1A and 1B, the vascular graft 10 comprises an elongated main body portion 12 having a first end 24 defining a first opening 26 and a second end 28 defining a second opening 30. The main body portion 12 includes a lumen 32 extending between the first and second ends 24 and 28 and being defined by an inner surface 34. The main body portion 12 can have any shape and size to facilitate surgical placement of the vascular graft 10 so that the main body portion conforms or substantially conforms to the inner surface of a bodily vessel. As shown in FIG. 1A, the main body portion 12 also includes an oval-shaped aperture 14 defined by a perimeter and having a first sewing ring 18 securely attached thereto. It will be appreciated that the main body portion 12 can include more than one aperture 14, and that the aperture can have any suitable shape (e.g., circular or rectangular).

The first and second ends 24 and 28 of the main body portion 12 include second and third sewing rings 20 and 22. As shown in FIGS. 1A and 1B, the second sewing ring 20 is securely attached to the first end 24 of the main body portion 12 adjacent the first opening 26, and the third sewing ring 22 is securely attached to the second end 28 of the main body portion adjacent the second opening 30. Each of the first, second, and third sewing rings 18, 20, and 22 can have a circular or oval-like shape and be adapted for suturing to a portion of a bodily vessel, such as a portion of the aorta. The first, second, and third sewing rings 18, 20, and 22 are securely attached to the main body portion 12 of the vascular graft 10 using any one or combination of known attachment means (e.g., staples, clips, sutures, adhesives, etc.). The first, second, and third sewing rings 18, 20, and 22 are comprised of any suitable biocompatible material including, for example, woven polyester, DACRON, TEFLON, PTFE and/or any one or combination of the biocompatible materials provided above.

As shown in FIGS. 1A and 1B, the vascular graft 10 also includes a multi-lumen branch graft 16. The multi-lumen branch graft 16 comprises a plurality of branch members 36 and an attachment portion 38 adapted for anastomosis with the first sewing ring 18. The attachment portion 38 can include a substantially flat or dome-shaped member capable of mating with the branch members 36. The attachment portion 38 is dimensioned to correspond to the dimensions of the first sewing ring 18. As shown in FIG. 1A, for example, the attachment portion 38 has an oval-like shape which corresponds to the oval-like shape of the first sewing ring 18. The multi-lumen branch graft 16 can be securely connected to the first sewing ring 18 using sutures, for example, or, alternatively, formed as an integral part of the main body portion 12.

The multi-lumen branch graft 16 includes at least two branch members 36. Each of the branch members 36 has a generally tubular shape and includes first and second ends 40 and 42. A lumen (not shown) extends between the first and second ends 40 and 42 of each of the branch members 36. Each of the first ends 40 of the branch members 36 is securely attached to the attachment portion 38 of the multi-lumen branch graft 16 using, for example, clips, sutures, staples, etc. When the multi-lumen branch graft 16 is securely attached to the main body portion 12 (FIG. 1B), each of the lumens of the branch members 36 is in fluid communication with the lumen 32 of the main body portion. Although the multi-lumen branch graft 16 shown in FIGS. 1A and 1B has three branch members 36, it will be appreciated that the multi-lumen branch graft can have four, five, or even more branch members. By adding or removing branch members 36 from the multi-lumen branch graft 16, the vascular graft 10 can be easily modified for implantation at any number of desired vascular locations.

The main body portion 12 of the vascular graft 10 can additionally include at least one expandable support member 44 operably secured within the lumen 32 of the main body portion (FIGS. 1C-E). The expandable support member 44 has oppositely disposed first and second end portions 46 and 48 and a main body portion 50 extending between the end portions. The main body portion 50 can include an aperture 51 defined by a perimeter; however, it will be appreciated that the expandable support member 44 can have any number of apertures.

The structure of the expandable support member 44 may be a mesh, a zigzag wire, diamond-shaped, a spiral wire, an expandable stent, or other similar configuration that allows the expandable support member to be collapsed and expanded. The expandable support member 44 can be comprised of a material having a high modulus of elasticity, including, for example, cobalt-nickel alloys (e.g., Elgiloy), titanium, nickel-titanium alloys (e.g., Nitinol), cobalt-chromium alloys (e.g., Stellite), nickel-cobalt-chromium-molybdenum alloys (e.g., MP35N), graphite, ceramic, stainless steel, and hardened plastics.

The expandable support member 44 may also be made of a radio-opaque material or include radio-opaque markers (not shown) to facilitate fluoroscopic visualization. Examples of radio-opaque materials are known in the art and can include, but are not limited to, gold, gallium, technetium, indium, strontium, iodine, barium, bromine and phosphorus-containing compounds. As described in more detail below, radio-opaque markers can be used to facilitate implantation of the vascular graft 10 in a bodily vessel.

An expanded configuration of the expandable support member 44 is shown in FIG. 1C. In the expanded configuration, the expandable support member 44 has a circular cross-sectional shape for conforming to the circular cross-sectional shape of the main body portion 12. The flexible and expandable properties of the expandable support member 44 facilitate delivery of the vascular graft 10 by, for example, providing a platform from which a multi-stage implant procedure can be conducted. By conforming to the shape of the main body portion 12, the expanded configuration of the expandable support member 44 also promotes movement of blood through the vascular graft 10 while maintaining lumen patency.

It will be appreciated that the vascular graft 10 can additionally or optionally include at least one conduit 43 adapted for connecting with a pump, such as a cardiopulmonary bypass (CPB) pump (not shown) to promote antegrade blood flow. The conduit 43 can have a tube-like configuration and can extend radially from the main body portion 12 of the vascular graft 10. The conduit 43 can have a lumen that communicates with the lumen 32 of the main body portion 12 so that blood may flow through the lumen of the conduit and into the vascular graft 10 when, for example, the conduit is connected to a CPB pump.

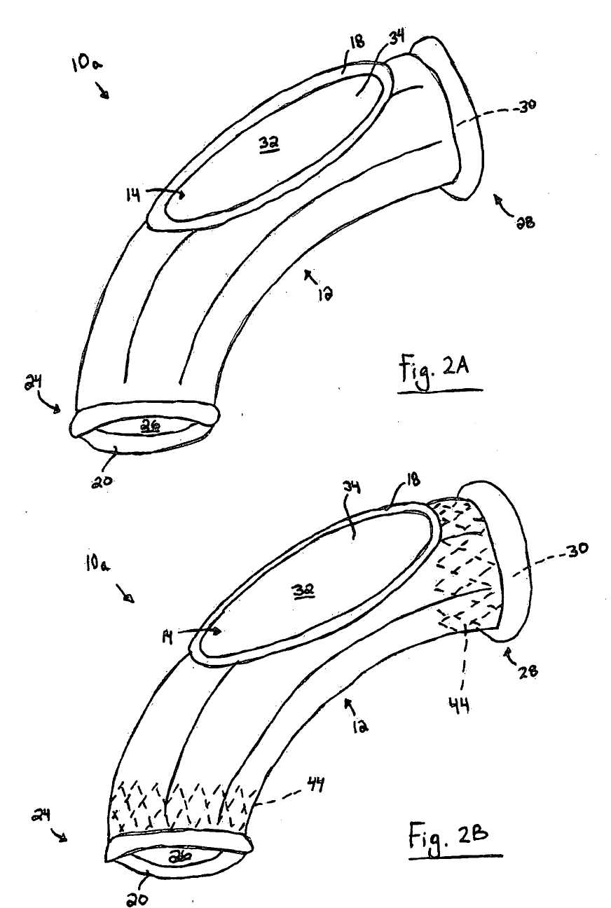

Another embodiment of the present invention is illustrated in FIGS. 2A and 2B. The vascular graft 10ₐ shown in FIGS. 2A and 2B is identically constructed as the vascular graft 10 shown in FIGS. 1A and 1B, except as described below. In FIGS. 2A and 2B, structures that are identical as structures in FIGS. 1A and 1B use the same reference numbers, whereas structures that are similar but not identical carry the suffix, “a”.

As shown in FIGS. 2A and 2B, the vascular graft 10ₐ can comprise an elongated main body portion 12 having a first end 24 defining a first opening 26 and a second end 28 defining a second opening 30. The main body portion 12 can also include an aperture 14 defined by a perimeter and having a first sewing ring 18 securely attached thereto. The first sewing ring 18 can be adapted for anastomosis with a portion of a native blood vessel, such as a portion of an aortic arch 110 (FIG. 28). For example, the first sewing ring 18 (FIGS. 2A and 2B) can be adapted for anastomosis with a portion of an aortic arch 110 (FIG. 28) including a brachiocephalic trunk artery 58, a left common carotid artery 60, and a left subclavian artery 62.

The vascular graft 10ₐ (FIGS. 2A and 2B) can further comprise a second sewing ring 20 securely attached to the first end 24 of the main body portion 12 adjacent the first opening 26, and a third sewing ring 22 securely attached to the second end 28 of the main body portion adjacent the second opening 30.

Referring to FIG. 2B, the main body portion 12 of the vascular graft 10ₐ can include at least one expandable support member 44. More particularly, each of the first and second ends 24 and 28 of the main body portion 12 can include an expandable support member 44. In an expanded configuration, the expandable support member 44 can have a circular cross-sectional shape for conforming to the circular cross-sectional shape of the main body portion 12. The flexible and expandable properties of the expandable support member 44 can facilitate delivery of the vascular graft 10ₐ by, for example, providing a platform from which a multi-stage implant procedure can be conducted. By conforming to the shape of the main body portion 12, the expanded configuration of the expandable support member 44 can also promote movement of blood through the vascular graft 10ₐ while maintaining lumen patency.

It will be appreciated that the vascular graft 10ₐ can additionally or optionally include at least one conduit (not shown) adapted for connecting with a pump, such as a CPB pump (not shown) to promote antegrade blood flow. The conduit can have a tube-like configuration and can extend radially from the main body portion 12 of the vascular graft 10ₐ. The conduit can have a lumen that communicates with the lumen 32 of the main body portion 12 so that blood may flow through the lumen of the conduit and into the vascular graft 10ₐ when, for example, the conduit is connected to a CPB pump.

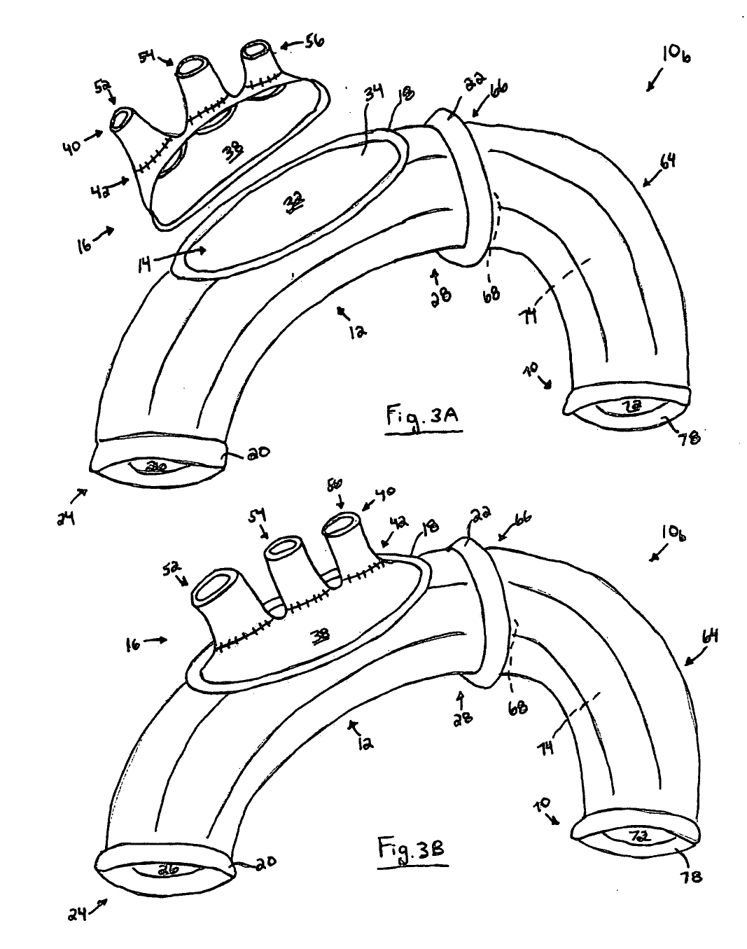

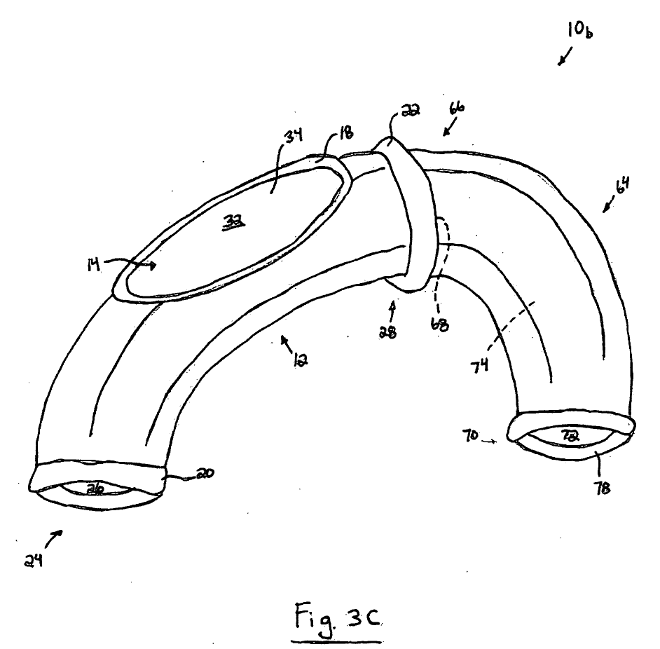

Another embodiment of the present invention is illustrated in FIGS. 3A-C. The vascular graft 106 shown in FIGS. 3A-C is identically constructed as the vascular graft 10 shown in FIGS. 1A and 1B, except as described below. In FIGS. 3A-C, structures that are identical as structures in FIGS. 1A and 1B use the same reference numbers, whereas structures that are similar but not identical carry the suffix “b”.

As shown in FIGS. 3A-C, the vascular graft 10_{b} can comprise an elongated main body portion 12 having a first end 24 defining a first opening 26 and a second end 28 defining a second opening 30. The main body portion 12 can also include an aperture 14 defined by a perimeter and having a first sewing ring 18 securely attached thereto. The vascular graft 10_{b} can further comprise a second sewing ring 20 securely attached to the first end 24 of the main body portion 12 adjacent the first opening 26, a third sewing ring 22 securely attached to the second end 28 of the main body portion adjacent the second opening 30, and a multi-lumen branch graft 16 adapted for anastomosis with the aperture 14 of the main body portion. The multi-lumen branch graft 16 can be securely connected to the first sewing ring 18 using sutures, for example, or, alternatively, formed as an integral part of the main body portion 12. It will be appreciated that the vascular graft 10_{b} can include any additional number of sewing rings 22.

The multi-lumen branch graft 16 can include first, second, and third branch members 52, 54, and 56 securely attached to an attachment portion 38. Each of the first, second, and third branch members 52, 54, and 56 can have a generally tubular configuration and include first and second ends 40 and 42. Each of the second ends 42 of the first, second, and third branch members 52, 54, and 56 can be respectively configured to anastomose or accommodate a brachiocephalic trunk artery 58 (FIG. 14), a left common carotid artery 60, and a left subclavian artery 62.

The vascular graft 10_{b} (FIGS. 3A-C) can also include a second main body portion 64 having a first end 66 defining a first opening 68 and a second end 70 defining a second opening 72. The second main body portion 64 can have an elongated, tube-like configuration and include a lumen 74 extending between the first and second ends 66 and 70 and being defined by an inner surface 76. The second main body portion 64 can have any shape and size to facilitate placement of the vascular graft 10_{b} so that the second main body portion conforms or substantially conforms to the inner surface of a bodily vessel. The second main body portion 64 can be made of a biocompatible material, such as woven polyester. DACRON, PTFE and/or TEFLON. The material used to construct the second main body portion 64 can be the same or nearly the same as the material used to construct the main body portion 12.

The second main body portion 64 can also include a fourth sewing ring 78 securely attached to the second end 70 of the second main body portion adjacent the second opening 72. As shown in FIGS. 3A-C, the fourth sewing ring 78 can have a circular shape adapted for anastomosis with a bodily vessel, such as the aorta. The fourth sewing ring 78 can be securely attached to the second end 70 of the second main body portion 64 using any one or combination of known attachment means (e.g., staples, sutures, clips, pins, adhesives, etc.). The fourth sewing ring 78 can be comprised of any suitable biocompatible material including, for example, woven polyester, DACRON, PTFE and/or TEFLON.

Referring to FIGS. 3A-C, the first end 66 of the second main body portion 64 can be securely attached to the third sewing ring 22 of the main body portion 12 or, alternatively, the main body portion itself. The second main body portion 64 can be securely attached to the main body portion 12 so that the lumen 74 of the second main body portion is in fluid communication with the lumen 32 of the main body portion. The third sewing ring 22 can enable repair of complex aortic lesions that involve both the aortic arch and the descending aorta, even in the presence of a size mismatch between the vascular graft 10_{b} and the aorta by covering the gap between the aorta and the vascular graft (e.g., during an elephant trunk procedure). The second main body portion 64 can be securely attached to the third sewing ring 22 using sutures, for example, or any other known attachment means (e.g., staples, clips, adhesives, etc.).

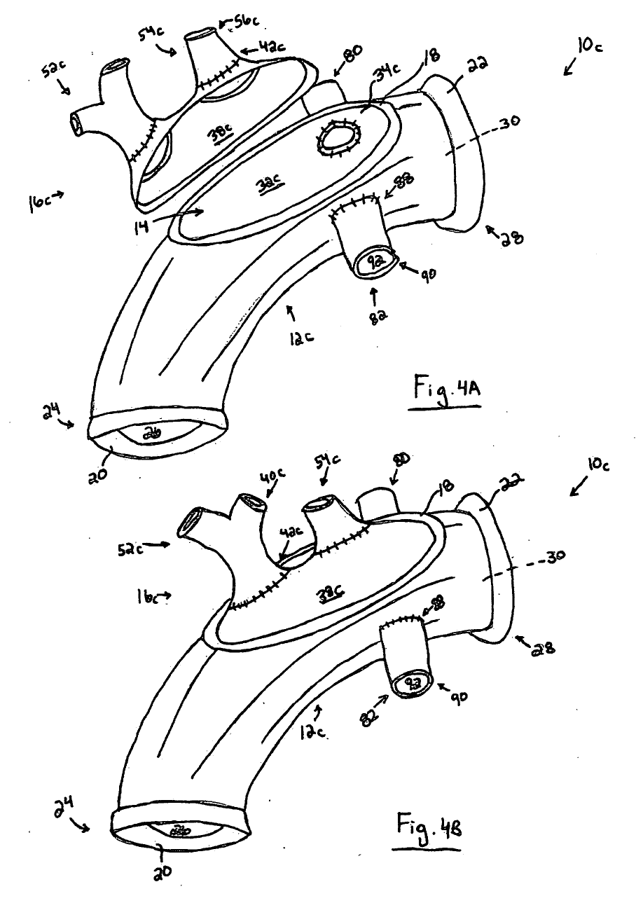

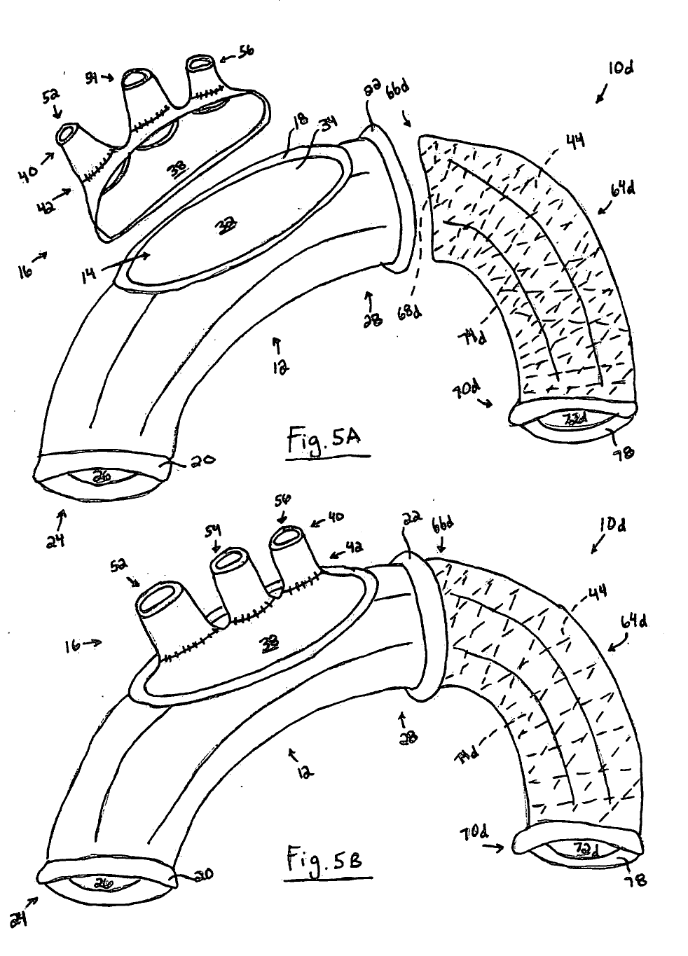

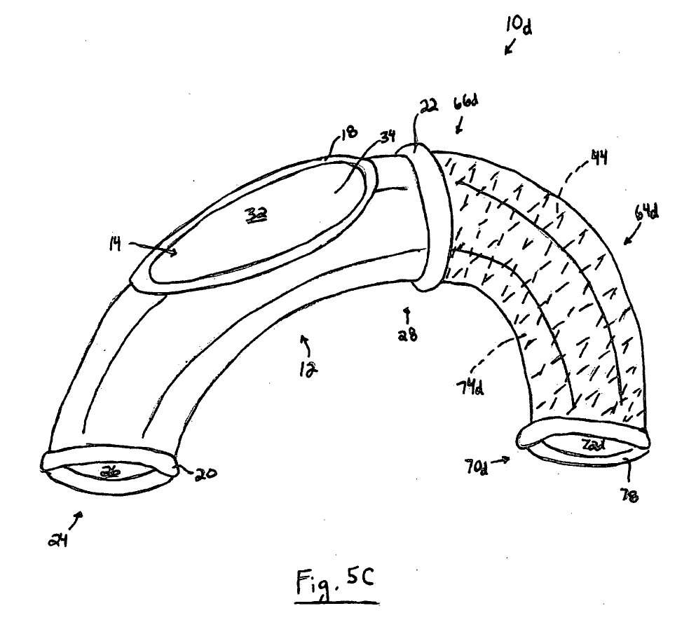

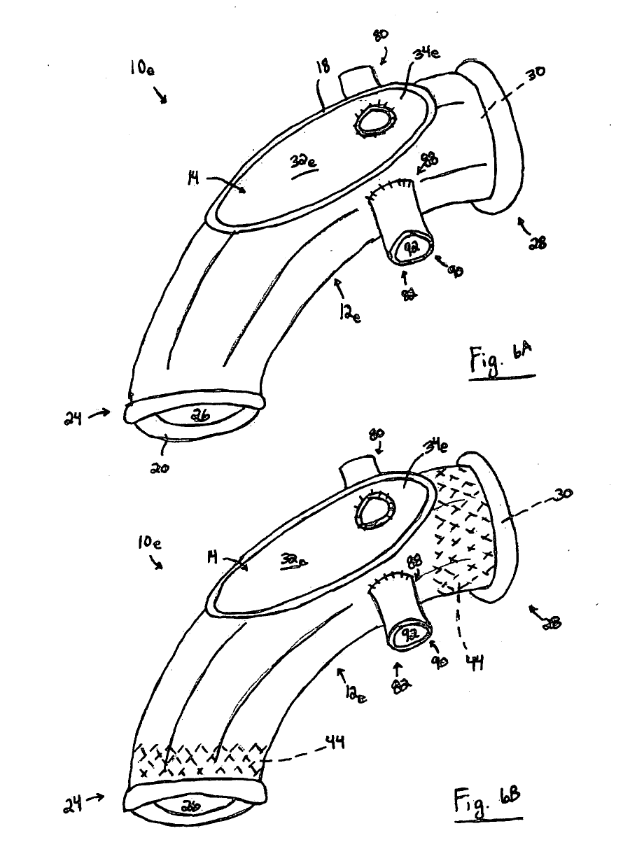

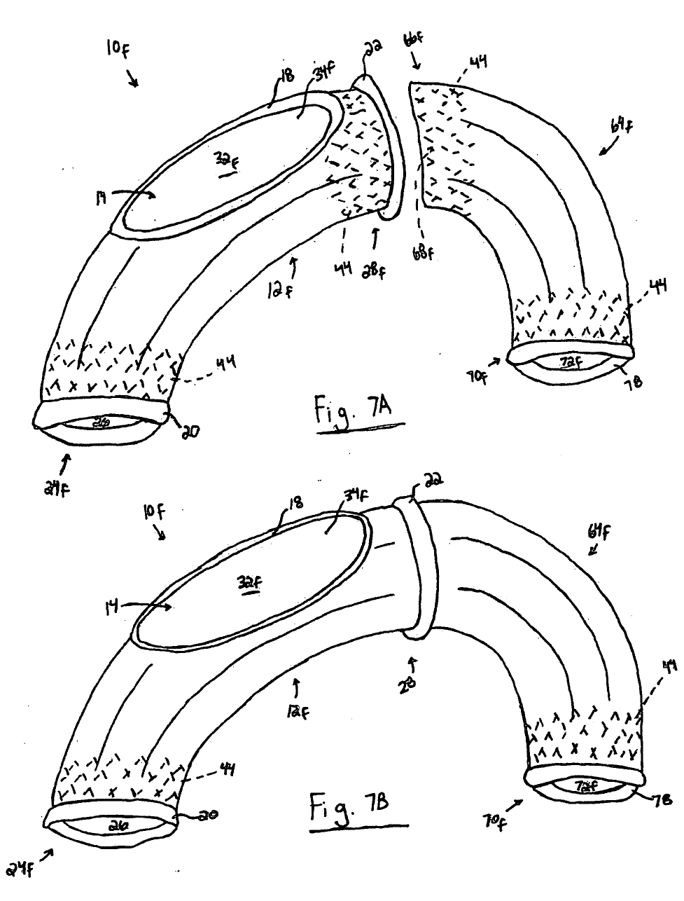

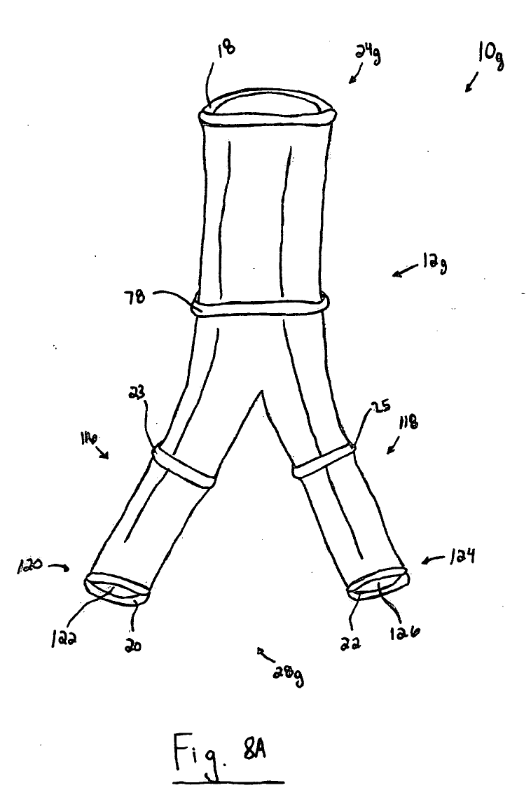

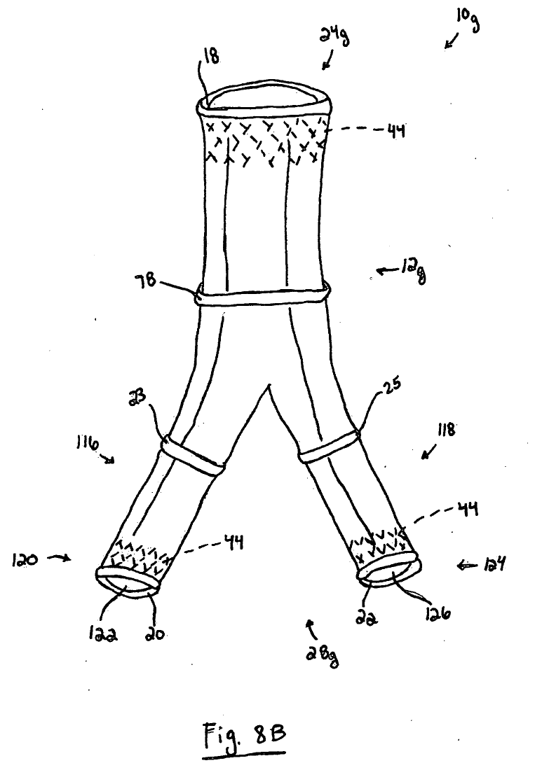

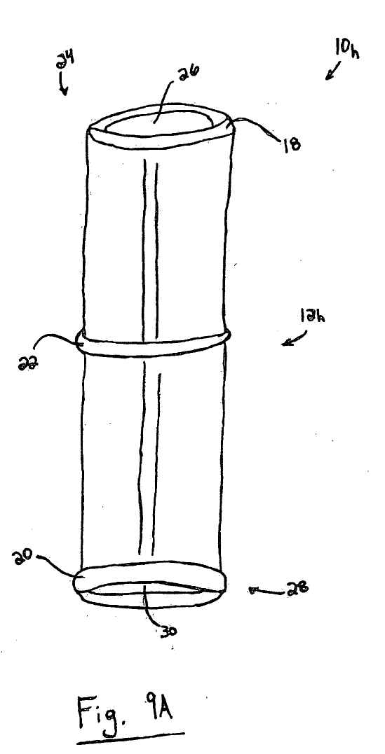

Another embodiment of the present invention is illustrated in FIGS. 4A and 4B. The vascular graft 10_{c} shown in FIGS. 4A and 4B is identically constructed as the vascular graft 10 shown in FIGS. 1A and 1B, except as described below. In FIGS. 4A and 4B, structures that are identical as structures in FIGS. 1A and 1B use the same reference numbers, whereas structures that are similar but not identical carry the suffix “c”.

As shown in FIGS. 4A and 4B, the vascular graft 10_{c} can comprise an elongated main body portion 12_{c} having a first end 24 defining a first opening 26 and a second end 28 defining a second opening 30. The main body portion 12_{c} can include a lumen 32_{c} extending between the first and second ends 24 and 28 and being defined by an inner surface 34_{c}. The main body portion 12_{c} can also include an aperture 14 defined by a perimeter and having a first sewing ring 18 securely attached thereto. The main body portion 12_{c} can also include first and second arm members 80 and 82 respectively configured to accommodate the left and right renal arteries 84 and 86 (FIG. 10).

The first and second arm members 80 and 82 (FIGS. 4A and 4B) can have a generally tube-like configuration and include first and second ends 88 and 90. Each of the first and second arm members 80 and 82 can also include a lumen 92 extending between the ends. The first end 88 of each of the first and second arm members 80 and 82 can be securely attached to the main body portion 12_{c} so that the lumen 92 of each of the arm members is in fluid communication with the lumen 32_{c} of the main body portion. Each of the second ends 90 of the first and second arm members 80 and 82 can be respectively configured to accommodate the left and right renal arteries 84 and 86. The first and second arm members 80 and 82 can be securely attached to the main body portion 12_{c} using sutures, for example. Alternatively, the first and second arm members 80 and 82 can be integrally formed with the main body portion 12. The first and second arm members 80 and 82 can be made of a biocompatible material, such as woven polyester, DACRON, PTFE and/or TEFLON.

As shown in FIGS. 4A and 4B, the multi-lumen branch graft 16_{c} can include first and second branch members 52_{c} and 54_{c} securely attached to an attachment portion 38_{c}. Each of the first and second branch members 52_{c} and 54_{c} can have a tubular configuration and include first and second ends 40_{c} and 42_{c}. The second end 42_{c} of the first branch member 52_{c} can have a bifurcated configuration to accommodate a celiac trunk 94 (FIG. 10), and the second end 42_{c} (FIGS. 4A and 4B) of the second branch member 54_{c} can be configured to accommodate a superior mesenteric artery 96 (FIG. 10). Each of the first ends 40_{c} (FIGS. 4A and 4B) of the first and second branch members 52_{c} and 54_{c} can be securely attached to the attachment portion 38_{c} of the multi-lumen branch graft 16_{c} using sutures, for example. The first and second branch members 52_{c} and 54_{c} can be made of a biocompatible material, such as woven polyester, DACRON, PTFE and/or TEFLON.

Another embodiment of the present invention is illustrated in FIGS. 5A-C. The vascular graft 10_{d} shown in FIGS. 5A-C is identically constructed as the vascular graft 10_{b} shown in FIGS. 3 A and 3B, except as described below. In FIGS. 5A-C, structures that are identical as structures in FIGS. 3A and 3B use the same reference numbers, whereas structures that are similar but not identical carry the suffix “d”.

As shown in FIGS. 5A-C, the vascular graft 10_{d} can comprise an elongated main body portion 12 having a first end 24 defining a first opening 26 and a second end 28 defining a second opening 30. The main body portion 12 can also include an aperture 14 defined by a perimeter and having a first sewing ring 18 securely attached thereto. The vascular graft 10_{d} can further comprise a second sewing ring 20 securely attached to the first end 24 of the main body portion 12 adjacent the first opening 26, a third sewing ring 22 securely attached to the second end 28 of the main body portion adjacent the second opening 30, and a multi-lumen branch graft 16 (FIGS. 5A and 5B) adapted for anastomosis with the aperture 14 of the main body portion.

The multi-lumen branch graft 16 can include first, second, and third branch members 52, 54, and 56 securely attached to an attachment portion 38. Each of the first, second, and third branch members 52, 54, and 56 can have a generally tubular configuration and include first and second ends 40 and 42. Each of the second ends 42 of the first, second, and third branch members 52, 54, and 56 can be respectively configured to accommodate a brachiocephalic trunk artery 58 (FIG. 14), a left common carotid artery 60, and a left subclavian artery 62.

The vascular graft 10_{d} (FIGS. 5A-C) can also include a second main body portion 64_{d} having a first end 66_{d} defining a first opening 68_{d} and a second end 70_{d} defining a second opening 72_{d}. The second main body portion 64_{d} can have an elongated, tube-like configuration and include a lumen 74_{d} extending between the first and second ends 66_{d} and 70_{d} and being defined by an inner surface 76_{d}. The second main body portion 64_{d} can have any shape and size to facilitate placement of the vascular graft 10_{d} so that the second main body portion conforms or substantially conforms to the inner surface of a bodily vessel. The second main body portion 64_{d} can be made of a biocompatible material, such as woven polyester, DACRON, PTFE and/or TEFLON. The second main body portion 64_{d} can be constructed of a material that is the same or substantially the same as the material used to construct the main body portion 12.

The second main body portion 64_{d} can include a fourth sewing ring 78 securely attached to the second end 70_{d} of the second main body portion. As shown in FIGS. 5A-C, the fourth sewing ring 78 can have a circular shape adapted for anastomosis with a bodily vessel, such as the aorta. The fourth sewing ring 78 can be securely attached to the second end 70_{d} of the second main body portion 64_{d} using any one or combination of known attachment means (e.g., staples, sutures, clips, pins, adhesives, etc.). The fourth sewing ring 78 can be comprised of any suitable biocompatible material including, for example, woven polyester, DACRON, PTFE and/or TEFLON. It will be appreciated that the vascular graft 10_{d} can include any additional number of sewing rings 22.

As illustrated in FIGS. 5A-C, the second main body portion 64_{d} can also include an expandable support member 44 operably disposed in the lumen 74_{d} of the second main body portion. The expandable support member 44 can comprise a stent, for example, similar or identical to the ones shown in FIGS. 1C and 1D. The expandable support member 44 can be made from a flexible, resiliently bendable material, such as Nitinol. As described above, the expandable support member 44 can include a radio-opaque marker (not shown) to facilitate placement of the vascular graft 10_{d}. The expandable support member 44 may also serve as a platform for multi-stage implant procedures.

Referring to FIGS. 5B and 5C, the first end 66_{d} of the second main body portion 64_{d} can be securely attached to the third sewing ring 22 of the main body portion 12 or, alternatively, to the main body portion itself. The second main body portion 64_{d} can be securely attached to the main body portion 12 so that the lumen 74_{d} of the second main body portion is in fluid communication with the lumen 32 of the main body portion. The third sewing ring 22 can enable repair of complex aortic lesions that involve both the aortic arch and the descending aorta even in the presence of a size mismatch between the vascular graft 10_{d} and the aorta, by covering the gap between the aorta and the vascular graft. The second main body portion 64_{d} can be securely attached to the third sewing ring 22 using sutures, for example, or any other known attachment means (e.g., staples, clips, adhesives, etc.).

Another embodiment of the present invention is illustrated in FIGS. 6A and 6B. The vascular graft 10_{c} shown in FIGS. 6A and 6B is identically constructed as the vascular graft 10_{c} shown in FIGS. 4A and 4B, except as described below. In FIGS. 6A and 6B, structures that are identical as structures in FIGS. 4A and 4B use the same reference numbers, whereas structures that are similar but not identical carry the suffix “e”.

As shown in FIGS. 6A and 6B, the vascular graft 10ₑ can comprise an elongated main body portion 12ₑ having a first end 24 defining a first opening 26 and a second end 28 defining a second opening 30. The main body portion 12ₑ can include a lumen 32ₑ extending between the first and second ends 24 and 28 and being defined by an inner surface 34ₑ. The main body portion 12ₑ can also include an aperture 14 defined by a perimeter and having a first sewing ring 18 securely attached thereto. The first sewing ring 18 can be adapted for anastomosis with a portion of a native blood vessel, such as a portion of an abdominal aorta 98 (FIG. 10). The portion of the abdominal aorta 98 can include at least one abdominal artery selected from the group consisting of a celiac trunk artery 94, a superior mesenteric artery 96, an inferior mesenteric artery (not shown), an inferior phrenic artery (not shown), a middle suprarenal artery (not shown), a gonadal artery (not shown), a lumbar artery (not shown), and a middle sacral artery (not shown). For example, the first sewing ring 18 (FIGS. 6A and 6B) can be adapted for anastomosis with a portion of an abdominal aorta 98 including a celiac trunk artery 94 and a superior mesenteric artery 96 (FIG. 10).

The main body portion 12ₑ (FIGS. 6A and 6B) can also include first and second arm members 80 and 82 respectively configured to accommodate the left and right renal arteries 84 and 86 (FIG. 10). The first and second arm members 80 and 82 (FIGS. 6A and 6B) can have a generally tube-like configuration and include first and second ends 88 and 90. The first and second arm members 80 and 82 can also include a lumen 92 extending between the ends. The first end 88 of each of the first and second arm members 80 and 82 can be securely attached to the main body portion 12, so that the lumen 92 of each of the arm members is in fluid communication with the lumen 32ₑ of the main body portion. The first and second arm members 80 and 82 can be securely attached to the main body portion 12ₑ using sutures, for example. The first and second arm members 80 and 82 can be made of a biocompatible material, such as woven polyester, DACRON, PTFE and/or TEFLON.

Referring to FIG. 6B, the main body portion 12ₑ of the vascular graft 10ₑ can include at least one expandable support member 44. More particularly, each of the first and second ends 24 and 28 of the main body portion 12ₑ can include an expandable support member 44. In an expanded configuration, the expandable support member 44 can have a circular cross-sectional shape for conforming to the circular cross-sectional shape of the main body portion 12ₑ. The flexible and expandable properties of the expandable support member 44 can facilitate delivery of the vascular graft 10ₑ by, for example, providing a platform from which a mufti-stage implant procedure can be conducted. By conforming to the shape of the main body portion 12ₑ, the expanded configuration of the expandable support member 44 can also promote movement of blood through the vascular graft 10ₑ while maintaining lumen patency.

Another embodiment of the present invention is illustrated in FIGS. 7A and 7B. The vascular graft 10_{f} shown in FIGS. 7A and 7B is identically constructed as the vascular graft 10_{d} shown in FIGS. 5A and 5B, except as described below. In FIGS. 7A and 7B, structures that are identical as structures in FIGS. 5A and 5B use the same reference numbers, whereas structures that are similar but not identical carry the suffix “f”.

As shown in FIGS. 7A and 7B, the vascular graft 10_{f} can comprise an elongated main body portion 12_{f} having a first end 24_{f} defining a first opening 26 and a second end 28_{f} defining a second opening 30. The main body portion 12_{f} can also include an aperture 14 defined by a perimeter and having a first sewing ring 18 securely attached thereto. The first sewing ring 18 can be adapted for anastomosis with a portion of a native blood vessel, such as a portion of an aortic arch 110 (FIG. 28). For example, the first sewing ring 18 (FIGS. 7A and 7B) can be adapted for anastomosis with a portion of an aortic arch 110 (FIG. 28) including a brachiocephalic trunk artery 58, a left common carotid artery 60, and a left subclavian artery 62.

The main body portion 12_{f} of the vascular graft 10_{f} shown in FIG. 7A can further include at least one expandable support member 44. More particularly, each of the first and second ends 24_{f} and 28_{f} of the main body portion 12_{f} can include an expandable support member 44. In an expanded configuration, the expandable support member 44 can have a circular cross-sectional shape for conforming to the circular cross-sectional shape of the main body portion 12_{f}. The flexible and expandable properties of the expandable support member 44 can facilitate delivery of the vascular graft 10_{f} by, for example, providing a platform from which a multi-stage implant procedure can be conducted. By conforming to the shape of the main body portion 12_{f}, the expanded configuration of the expandable support member 44 can also promote movement of blood through the vascular graft 10_{f} while maintaining lumen patency.

The vascular graft 10_{f} can further comprise a second sewing ring 20 securely attached to the first end 24_{f} of the main body portion 12_{f} adjacent the first opening 26, and a third sewing ring 22 securely attached to the second end 28_{f} of the main body portion adjacent the second opening 30. Referring to FIGS. 7A and 7B, the vascular graft 10_{f} can also include a second main body portion 64_{f} having a first end 66_{f} defining a first opening 68_{f} and a second end 70_{f} defining a second opening 72_{f}. The second main body portion 64_{f} can have an elongated, tube-like configuration and include a lumen 74_{f} extending between the first and second ends 66_{f} and 70_{f} and being defined by an inner surface 76_{f}. It will be appreciated that the vascular graft 10_{f} can include any additional number of sewing rings 22.

The second main body portion 64_{f} can have any shape and size to facilitate placement of the vascular graft 10_{f} so that the second main body portion conforms or substantially conforms to the inner surface of a bodily vessel. The second main body portion 64_{f} can be made of a biocompatible material, such as woven polyester, DACRON, PTFE and/or TEFLON. The second main body portion 64_{f} can be constructed of a material that is the same or substantially the same as the material used to construct the main body portion 12_{f}.

The second main body portion 64_{f} can include a fourth sewing ring 78 securely attached to the second end 70_{f} of the second main body portion. As shown in FIGS. 7A and 7B, the fourth sewing ring 78 can have a circular shape adapted for anastomosis with a bodily vessel, such as the aorta. The fourth sewing ring 78 can be securely attached to the second end 70_{f} of the second main body portion 64_{f} using any one or combination of known attachment means (e.g., staples, sutures, clips, pins, adhesives, etc.). The fourth sewing ring 78 can be comprised of any suitable biocompatible material including, for example, woven polyester. DACRON, PTFE and/or TEFLON.

As illustrated in FIGS. 7A and 7B, the second main body portion 64_{f} can also include an expandable support member 44 operably disposed in the lumen 74_{f} of the second main body portion. The expandable support member 44 can comprise a stent, for example, similar to the one shown in FIG. 1C. As shown in FIG. 7A, each of the first and second ends 66_{f} and 70_{f} of the second main body portion 64_{f} can include an expandable support member 44. Alternatively, only the second end 70_{f} of the second main body portion 64_{f} can include an expandable support member 44 (FIG. 7B). The expandable support member 44 can be made from a flexible, resiliently bendable material, such as Nitinol. As described above, the expandable support member 44 can include a radio-opaque marker (not shown) to facilitate placement of the vascular graft 10_{f}. The expandable support member 44 may also serve as a platform for multi-stage implant procedures.

Referring to FIG. 7B, the first end 66_{f} of the second main body portion 64_{f} can be securely attached to the third sewing ring 22 of the main body portion 12_{f} or, alternatively, the main body portion itself. The second main body portion 64_{f} can be securely attached to the main body portion 12_{f} SO that the lumen 74_{f} of the second main body portion is in fluid communication with the lumen 32 of the main body portion. The third sewing ring 22 can enable repair of complex aortic lesions that involve both the aortic arch 110 and the descending aorta 114, even in the presence of a size mismatch between the vascular graft 10_{f} and the aorta by covering the gap between the aorta and the vascular graft (e.g., using an elephant trunk procedure). The second main body portion 64_{f} can be securely attached to the third sewing ring 22 using sutures, for example, or any other known attachment means (e.g., staples, clips, adhesives. etc.).

Another embodiment of the present invention is illustrated in FIGS. 8A and 8B. The vascular graft 10_{g} shown in FIGS. 8A and 8B is identically constructed as the vascular graft 10 shown in FIGS. 1A and 1B, except as described below. In FIGS. 8A and 8B, structures that are identical as structures in FIGS. 1A and 1B use the same reference numbers, whereas structures that are similar but not identical carry the suffix “g”.

As shown in FIG. 8A, the vascular graft 10_{g} can comprise a tubular main body portion 12_{g} having a first end 24_{g} and a second end 28_{g}. The first end 24_{g} of the main body portion 12_{g} can define a first opening 26_{g} having a first sewing ring 18 securely attached thereto. The first sewing ring 18 can be adapted for anastomosis with a first portion of a descending abdominal aorta 114. It will be appreciated that the vascular graft 10_{g} can include any additional number of sewing rings 22.

The second end 28_{g} of the vascular graft 10_{g} can include tubular first and second leg members 116 and 118 fluidly connected to the main body portion 12_{g}. As shown in FIG. 8A, the first leg member 116 can include an end 120 defined by an opening 122, and the second leg member 118 can include an end 124 defined by an opening 126. The end 120 of the first leg member 116 can include a second sewing ring 20 adapted for anastomosis with a right iliac artery (not shown) or right femoral artery (not shown), and the end 124 of the second leg member 118 can include a third sewing ring 22 adapted for anastomosis with a left iliac artery (not shown) or left femoral artery (not shown). A fourth sewing ring 78 can also be securely connected to the main body portion 12A, intermediate the first end 24_{g} and the second end 28_{g}. It should be appreciated that fifth and sixth sewing rings 23 and 25 may also be securely attached at an intermediate portion of the first and second leg members 116 and 118 (respectively).

The vascular graft 10_{g} can include at least one expandable support member 44. As shown in FIG. 8B, for example, each of the ends 24_{g}, 120, and 124 can include an expandable support member 44 operably disposed therein. Each of the expandable support members 44 can comprise a stent, for example, similar to the one shown in FIG. 1D. The expandable support member 44 can be made from a flexible, resiliently bendable material, such as Nitinol. As described above, the expandable support member 44 can include a radio-opaque marker (not shown) to facilitate placement of the vascular graft 10_{g}. The expandable support member 44 may also serve as a platform for multi-stage implant procedures.

Another embodiment of the present invention is illustrated in FIGS. 9A and 9B. The vascular graft 10ₕ shown in FIGS. 9A and 9B is identically constructed as the vascular graft 10 shown in FIGS. 1A and 1B, except as described below. In FIGS. 9A and 9B, structures that are identical as structures in FIGS. 1A and 1B use the same reference numbers, whereas structures that are similar but not identical carry the suffix “h”.

Referring to FIG. 9A, the vascular graft 10ₕ can comprise an elongated main body portion 12ₕ having a first end 24 defining a first opening 26 and a second end 28 defining a second opening 30. The main body portion 12ₕ can have any shape and size to facilitate surgical placement of the vascular graft 10ₕ so that the main body portion conforms or substantially conforms to the inner surface of a bodily vessel, such as an abdominal aorta 98, as well as any one of the peripheral vessels (not shown).

The first and second ends 24 and 28 of the main body portion 12ₕ can include first, second, and third sewing rings 18, 20 and 22. As shown in FIG. 9A, the first sewing ring 18 can be securely attached to the first end 24 of the main body portion 12ₕ adjacent the first opening 26, the second sewing ring 20 can be securely attached to the second end 28 of the main body portion adjacent the second opening 30, and the third sewing ring 22 can be securely attached to the main body portion intermediate the first and second sewing rings. Each of the first, second, and third sewing rings 18, 20, and 22 can have a circular or oval-like shape and be adapted for suturing to a portion of a bodily vessel, such as a portion of the abdominal aorta 98 and/or any of the peripheral arteries (not shown).

The first, second, and third sewing rings 18, 20, and 22 can be securely attached to the main body portion 12ₕ of the vascular graft 10ₕ using any one or combination of known attachment means (e.g., staples, clips, sutures, adhesives, etc.). The first, second, and third sewing rings 18, 20, and 22 can be comprised of any suitable biocompatible material including, for example, woven polyester, DACRON, PTFE, TEFLON, and/or any one or combination of the biocompatible materials provided above. It will be appreciated that the vascular graft 10ₕ can include any additional number of sewing rings 22.

The vascular graft 10ₕ can include at least one expandable support member 44. As shown in FIG. 9B, for example, each of the first and second ends 24 and 28 can include an expandable support member 44 operably disposed therein. Each of the expandable support members 44 can comprise a stent, for example, similar to the one shown in FIG. 1D. The expandable support member 44 can be made from a flexible, resiliently bendable material, such as Nitinol. As described above, the expandable support member 44 can include a radio-opaque marker (not shown) to facilitate placement of the vascular graft 10ₕ. The expandable support member 44 may also serve as a platform for multi-stage implant procedures.

The present invention further provides a method for repairing a portion of an abdominal aorta 98 (FIG. 10) in a subject. As indicated by the shaded region in FIG. 10, the portion of the abdominal aorta 98 to be repaired can include an abdominal aortic aneurysm (AAA). AAA is a localized dilatation of the abdominal aorta 98 that exceeds the normal diameter. AAA is caused by a degenerative process of the aortic wall whose exact etiology remains unknown. AAA is most commonly located infrarenally; however, other locations, such as suprarenally. pararenally or thoraco-abdominally are also possible. To repair the AAA, a vascular graft 10_{c} as illustrated in FIGS. 4A and 4B is first provided. Next, a placement position for the vascular graft 10_{c} in the abdominal aorta 98 is determined using one or a combination of known imaging techniques, such as ultrasonography, fluoroscopy, angiography, CT, helical CT, CT angiogram, MRI, and/or MR angiography. After identifying the placement position, the subject is prepared for surgery. Although implantation of the vascular graft 10_{c} is described below using an open-abdominal surgical approach, it will be appreciated that other methods for implanting the vascular graft, such as a percutaneous or minimally invasive surgical technique may also be used.

Prior to implantation of the vascular graft 10_{c}, the vascular graft is loaded into a delivery sheath 100 (FIG. 12A) to facilitate delivery of the vascular graft. The delivery sheath 100 maintains the vascular graft 10_{c} in a sterile environment while also keeping the vascular graft in a compressed configuration prior to implantation. Although not shown in detail, the delivery sheath 100 comprises first and second envelope members 102 and 104 capable of containing respective portions of the vascular graft 10_{c} in a compressed configuration. Each of the first and second envelope members 102 and 104 includes a release mechanism 106 (or any other suitable alternative) for selectively releasing the vascular graft 10_{c} from the delivery sheath 100. For example, the release mechanism 106 can comprise a zipper (not shown in detail).

As shown in FIG. 12A, the release mechanism 106 includes at least one string 108 or line which, when pulled or retracted, causes the mechanism to separate each of the first and second envelope members 102 and 104 and thereby release the respective portions of the vascular graft 10_{c} from the envelope members. For example, the string 108 or line can be operably connected to a zipper-like release mechanism 106. The delivery sheath 100 can be made of a transparent, biocompatible material (e.g., a plastic polymer) to facilitate visualization of the vascular graft 10_{c} during implantation. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{c} to a desired location.

After loading the vascular graft 10_{c} into the delivery sheath 100, an incision (not shown) is made over the skin of the subject and through the muscle (not shown) overlying the abdominal aorta 98. The abdominal tissue (not shown) surrounding the abdominal aorta 98 is then manipulated to clearly expose the AAA. Next, the vessels superior and inferior to the AAA are tied off or clamped (not shown) to temporarily stop blood flow through the abdominal aorta 98. For example, portions of the abdominal aorta 98 both superior and inferior to the AAA are clamped. Additionally, portions of the left and right renal arteries 84 and 86, as well as the celiac trunk 94 and superior mesenteric artery 96 are clamped to temporarily prevent blood flow through the AAA. After clamping the vessels surrounding AAA, the diseased portion of the abdominal aorta 98 is resected as shown in FIG. 11.

Next, the delivery sheath 100 containing the vascular graft 10_{c} is positioned over the abdominal aorta 98 as shown in FIG. 12A. More particularly, the first end 24 of the vascular graft 10_{c} is positioned adjacent the abdominal aorta 98 superior to the left and right renal arteries 84 and 86, and the second end 28 of the vascular graft is positioned adjacent the abdominal aorta inferior to the renal arteries. Next, the string 108 or line of each of the first and second envelope members 102 and 104 is manipulated (e.g., pulled) so that the first and second envelope members begin to release the vascular graft 10_{c}. As the first and second envelope members 102 and 104 release the vascular graft 10_{c}, the main body portion 12_{c} begins to expand into the AAA (FIG. 12B). Upon completely removing the delivery sheath 100 from the vascular graft 10_{c}, the second sewing ring 20 is anastomosed with the superior portion of the abdominal aorta 98 (FIG. 12C). Next, the third sewing ring 22 is anastomosed with the inferior portion of the abdominal aorta 98 (FIG. 12D).

After stitching the second and third sewing rings 20 and 22 to the abdominal aorta 98 as shown in FIG. 12D, the left and right renal arteries 84 and 86 are respectively anastomosed with the first and second arm members 80 and 82 of the vascular graft 10_{c}. Next, the celiac trunk 94 and the superior mesenteric artery 96 are respectively anastomosed with the first and second branch members 52_{c} and 54_{c}. After the vascular graft 10_{c} is securely positioned in the abdominal aorta 98 (FIG. 13), the clamps are removed and normal blood flow can resume through the vascular graft. To complete the surgery, the abdominal tissue is returned to its place over the abdominal aorta 98 and the incision is closed with sutures.

In another embodiment of the present invention, a method is provided for repairing a thoracic aortic aneurysm, such as an aneurysm of the aortic arch 110 (FIG. 14) in a subject. To repair an aortic arch aneurysm, for example, an open-chest elephant trunk procedure using the vascular graft 10_{b} shown in FIGS. 3A-3C can be employed. Although implantation of the vascular graft 10_{b} is described below using an open surgical approach, it will be appreciated that other methods for implanting the vascular graft, such as a percutaneous or minimally invasive surgical technique may also be used.

To repair the aortic arch aneurysm, the vascular graft 10_{b} illustrated in FIGS. 3A and 3B can first be provided. Next, a placement position for the vascular graft 10_{b} in the aortic arch aneurysm can be determined using a known imaging technique, such as fluoroscopy, angiography, ultrasonography, CT, helical CT, CT angiogram, MRI, and/or MR angiography.

Prior to implanting the vascular graft 10_{b}, the vascular graft can be loaded into a delivery sheath 100 (FIG. 14) to facilitate delivery of the vascular graft. The delivery sheath 100 can maintain the vascular graft 10_{b} in a sterile environment while also keeping the vascular graft in a compressed configuration prior to implantation. As shown in FIG. 14, the delivery sheath 100 can be constructed in an identical or similar manner as described above. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{b} to a desired location.

After loading the vascular graft 10_{b} into the delivery sheath 100, the delivery sheath 100 containing the vascular graft 10_{b} can be inserted into the aortic arch 110 via an incision. As shown in FIG. 14, the second main body portion 64 of the vascular graft 10_{b} can be positioned in the descending aorta 112, and the third sewing ring 22 can be positioned over the proximal portion 114 of the descending aorta. After positioning the second main body portion 64 in the descending aorta 112, the string 108 or line of the second envelope member 104 can be manipulated (e.g., pulled) so that the second envelope member releases the second main body portion and the second main body portion expands into the descending aorta (FIG. 15). The fourth sewing ring 78 of the vascular graft 10_{b} can then be stitched to the descending aorta 112 (FIG. 17) or to another vascular structure as part of a second-stage elephant trunk procedure.

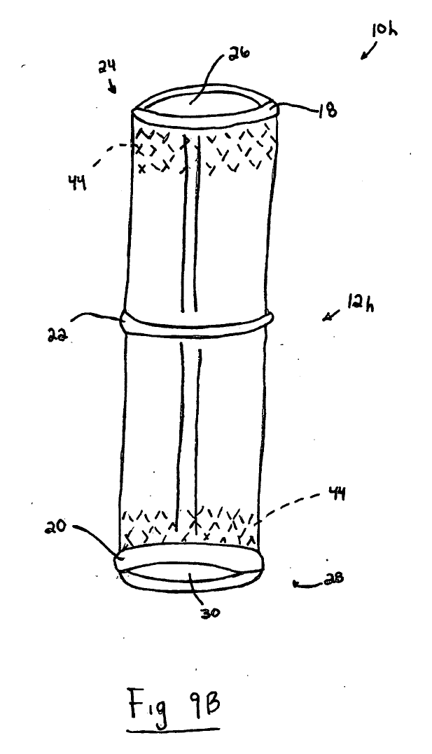

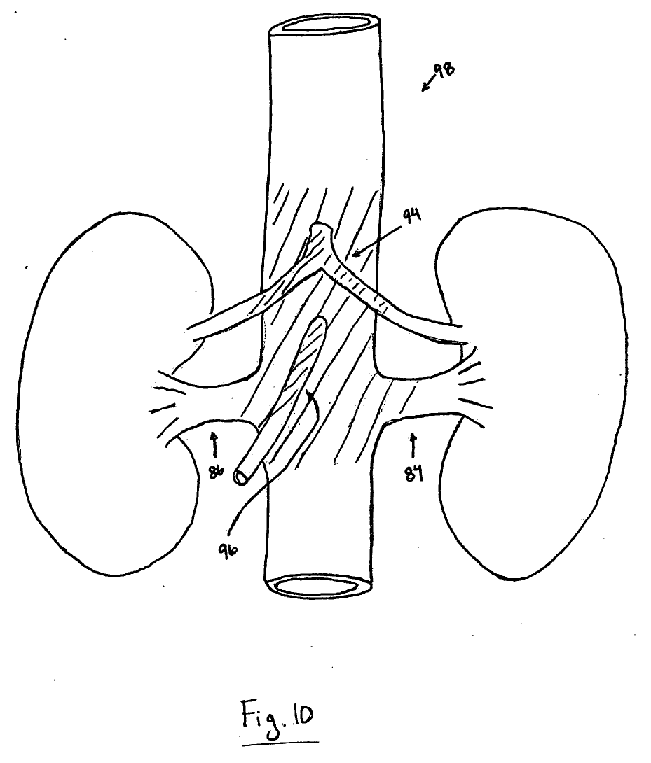

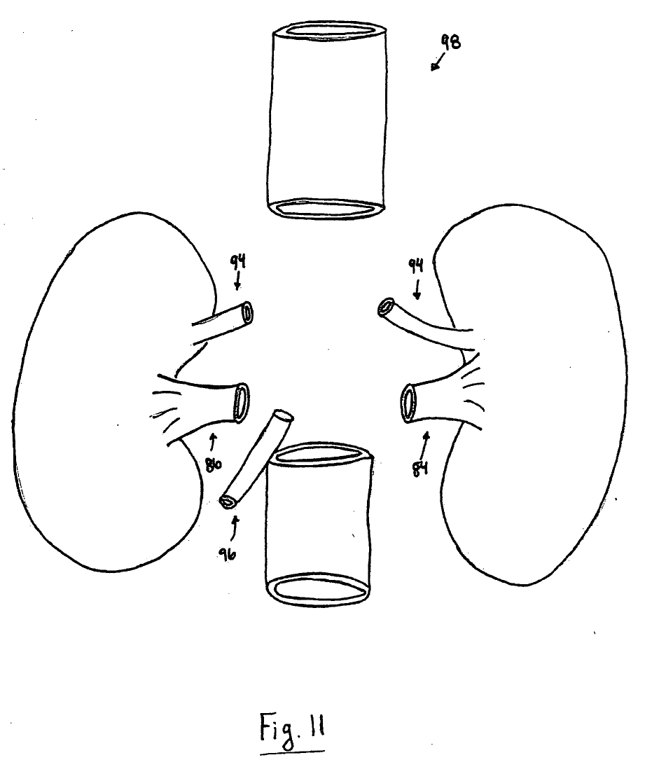

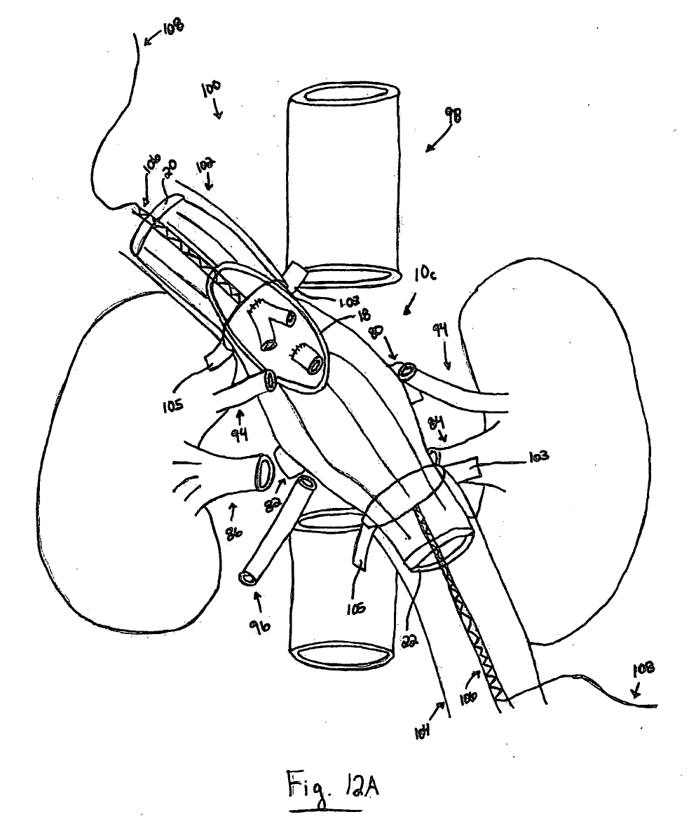

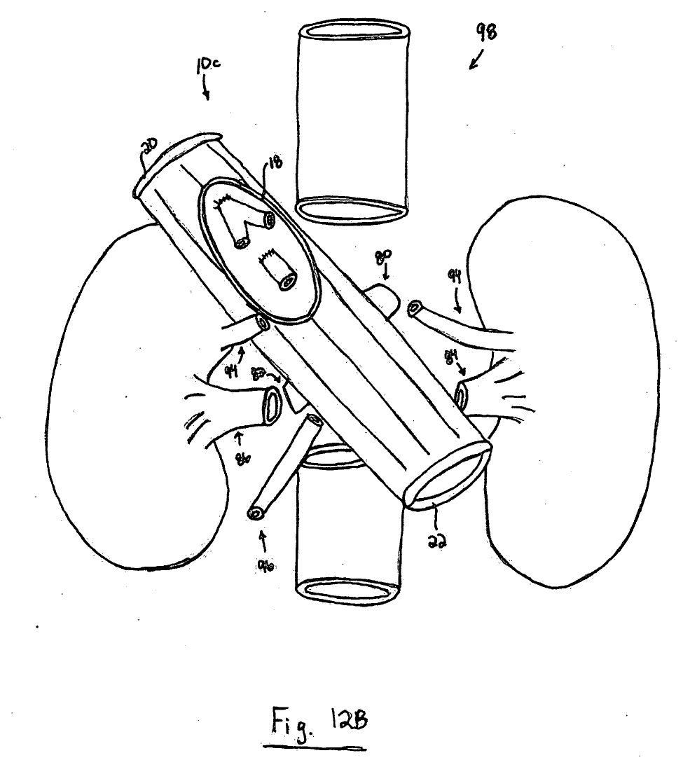

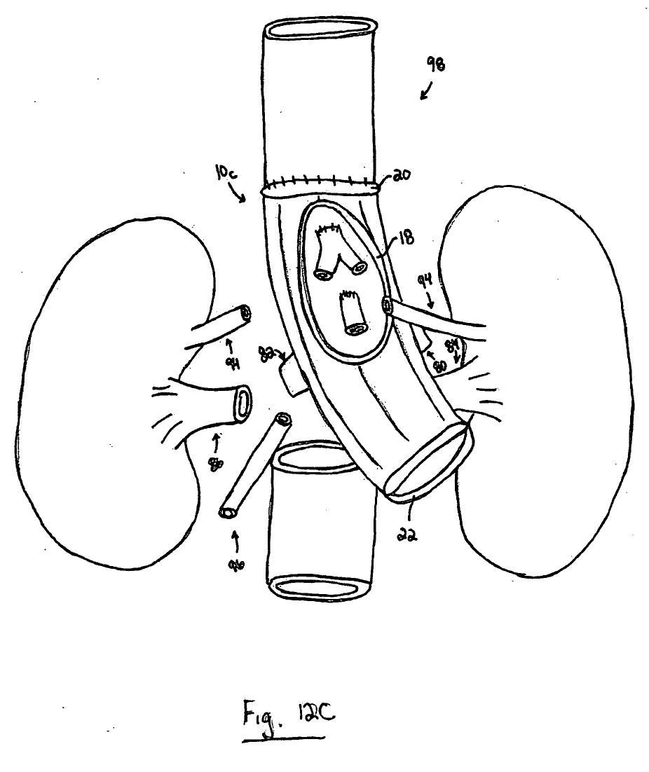

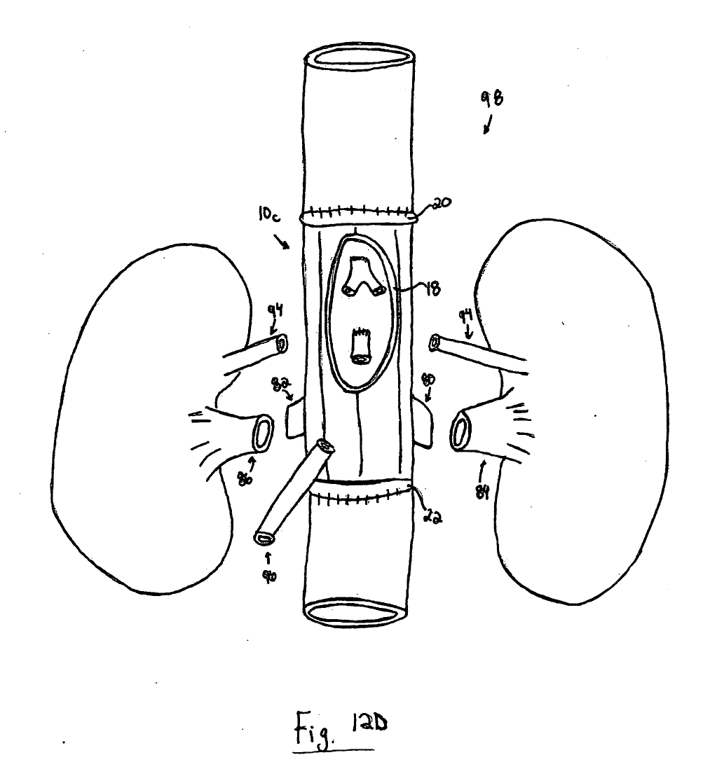

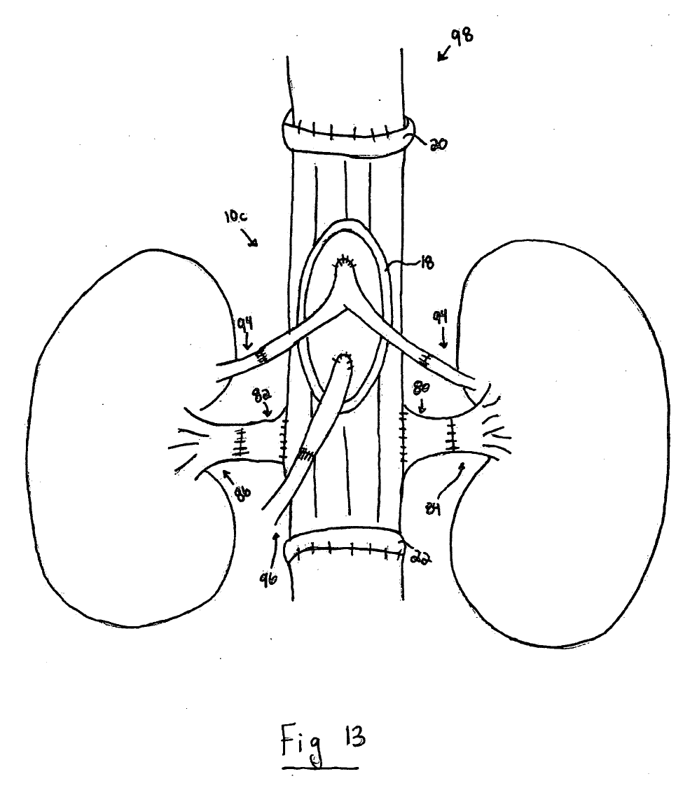

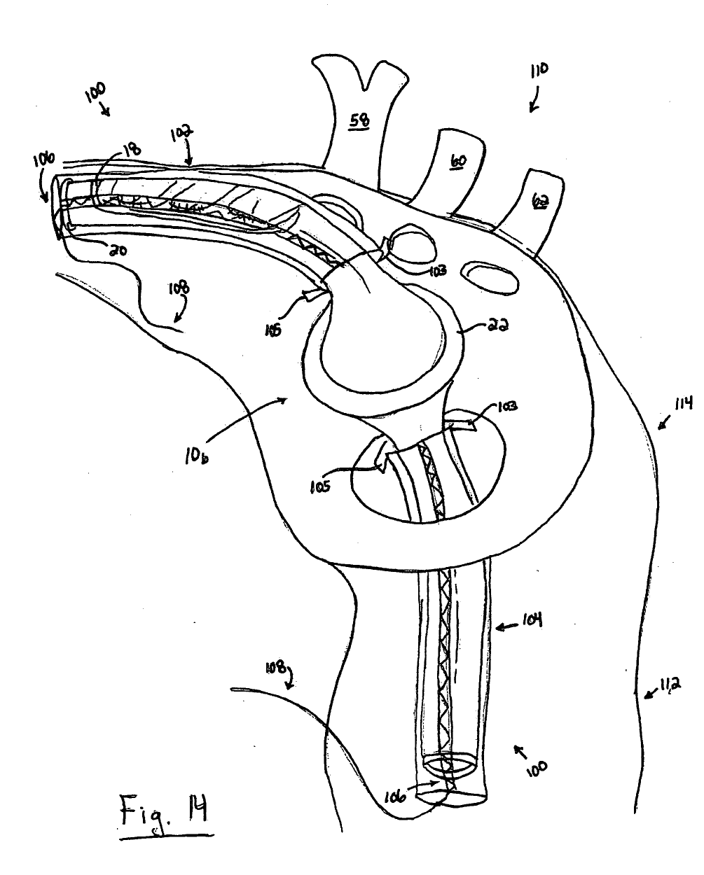

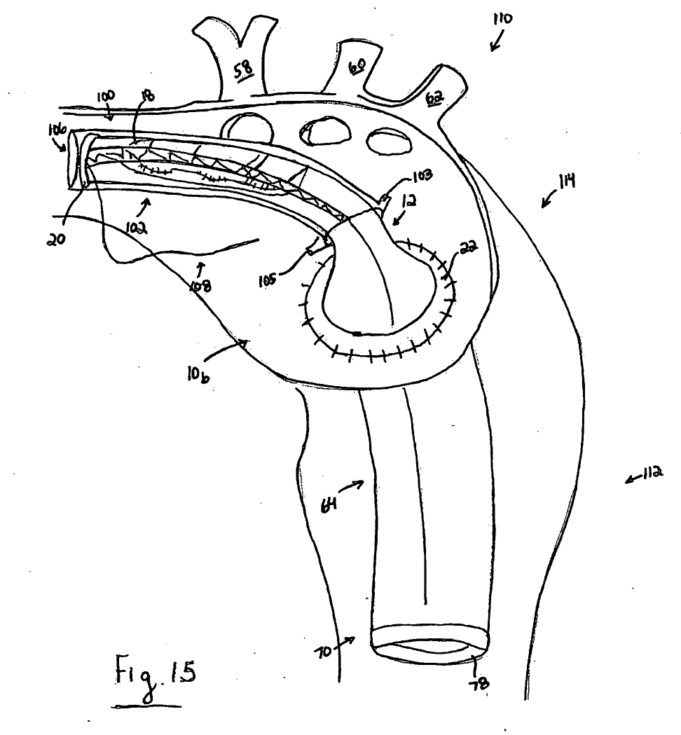

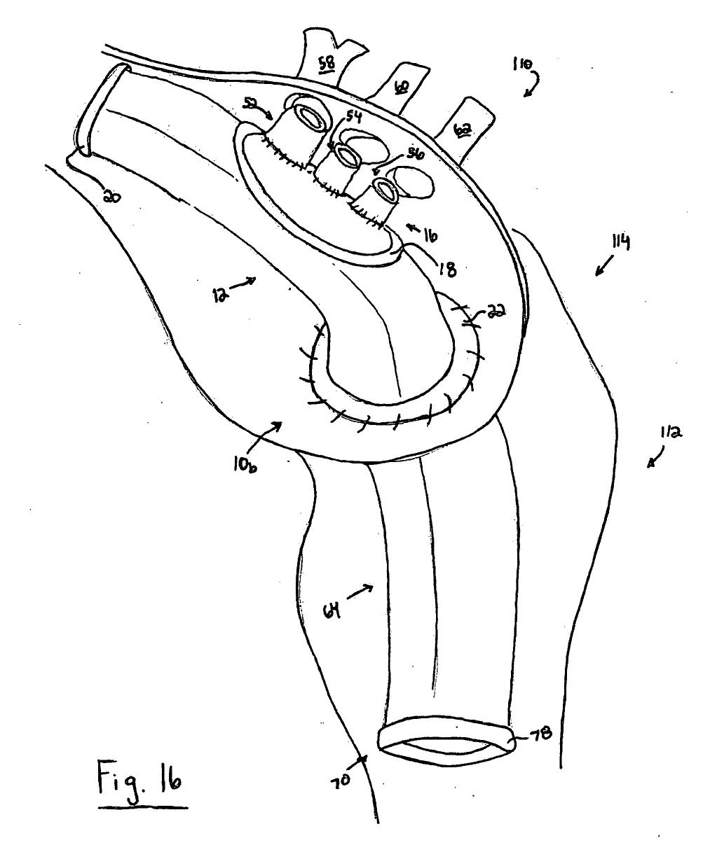

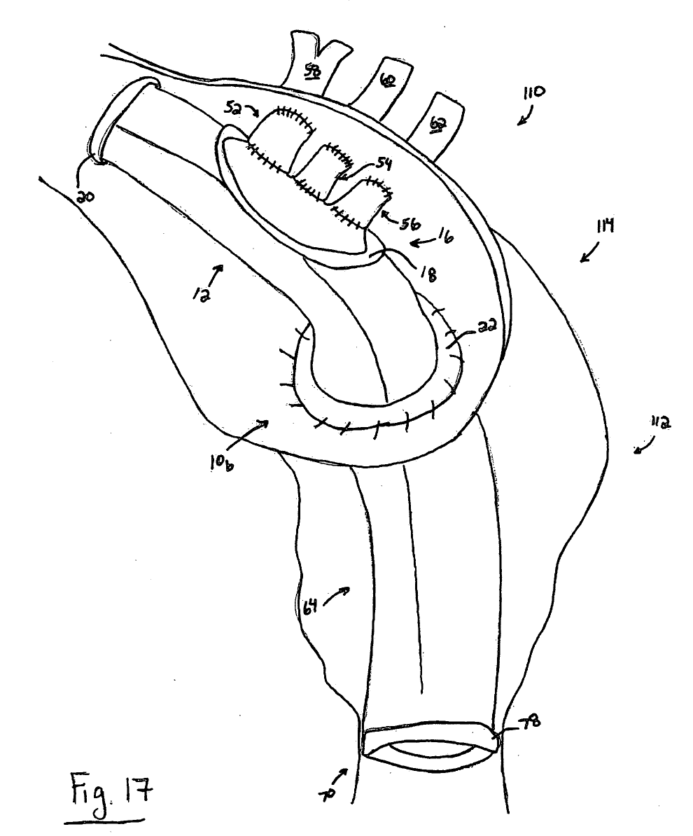

After securely attaching the third sewing ring 22 to the proximal portion 114 of the descending aorta 112, the string 108 or line of the first envelope member 102 can be manipulated (e.g., pulled) so that the first envelope member releases the main body portion 12 and the main body portion expands into the aortic arch 110 (FIG. 16). As shown in FIG. 17, the first, second and third branch members 52, 54, and 56 of the multi-lumen branch graft 16 can then be respectively anastomosed with the brachiocephalic trunk artery 58, the left common carotid artery 60, and the left subclavian artery 62. Although not shown in FIG. 17, the second sewing ring 20 of the vascular graft 10_{b} can then be secured to the aortic arch 110. It will be appreciated that the second sewing ring 20 can also be secured to the ascending aorta (not shown in detail), the aortic root (not shown), or used to secure a mechanical or biological bioprosthetic valve (not shown) in a native cardiac valve (not shown). After the vascular graft 10_{b} is secured in place of the aortic arch aneurysm, the incision in the aortic arch 110 can be closed and the vessels surrounding the vascular graft undamped so that blood can flow normally through the vascular graft.

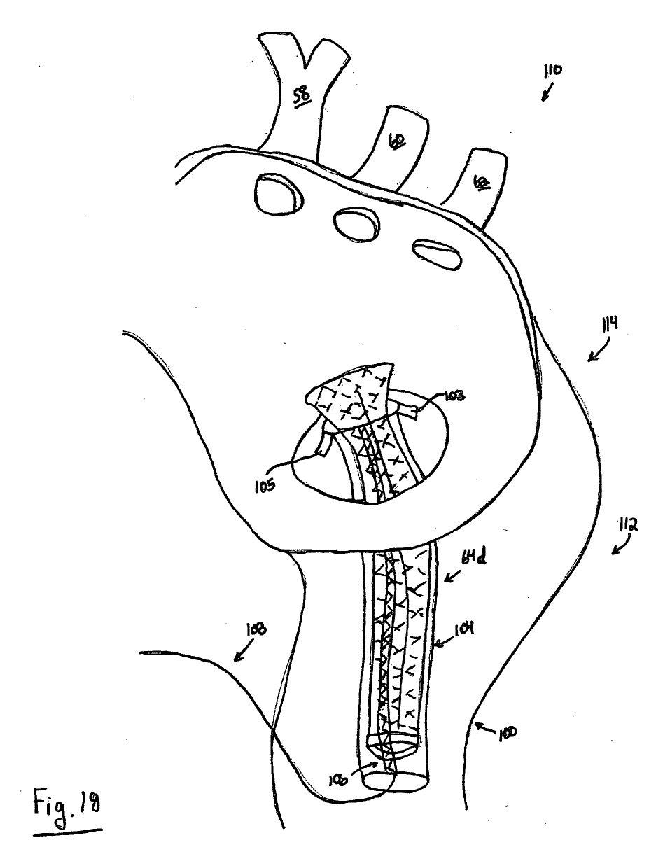

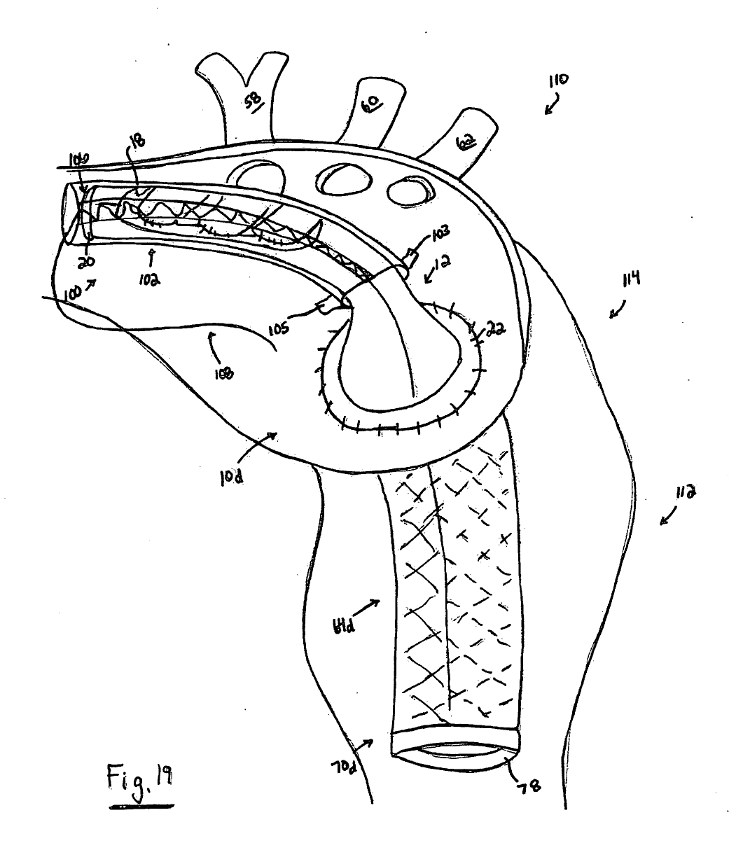

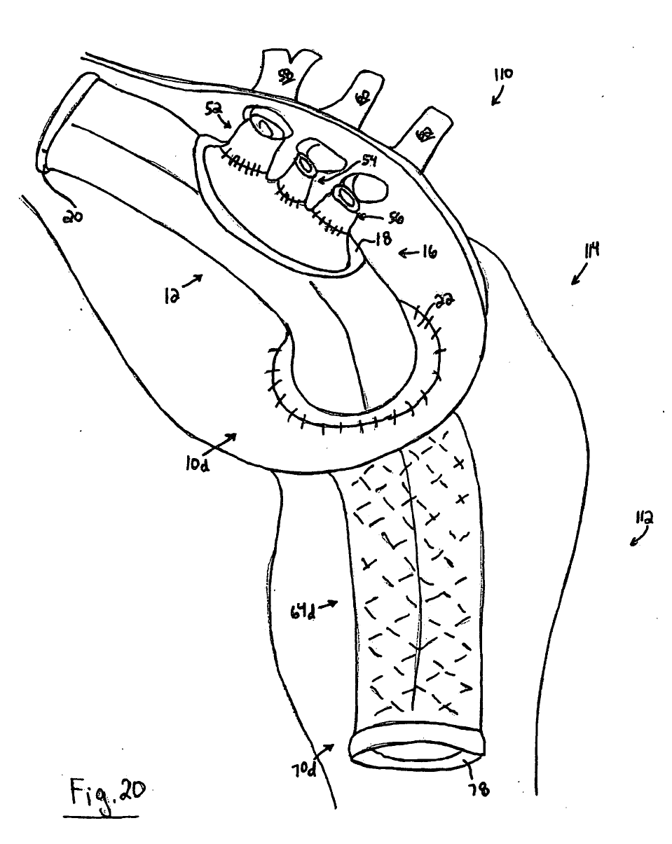

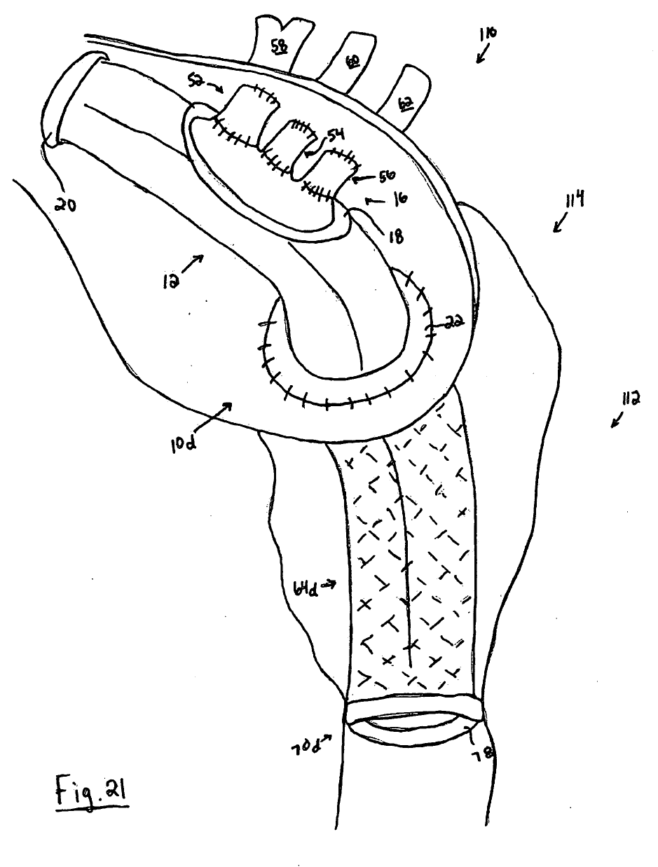

In another embodiment of the present invention, a method is provided for repairing a portion of an aortic arch 110 (FIG. 18) in a subject using, for example, a multi-stage approach. The multi-stage approach can be performed via an open-chest elephant trunk procedure to repair an aortic arch aneurysm. It will be appreciated, however, that other surgical approaches, such as a minimally invasive or percutaneous approach may also be used.

To repair the aortic arch 110 using a multi-stage approach (i.e., an elephant trunk procedure), a vascular graft 10_{d}, such as the one illustrated in FIGS. 5A and 5B can first be provided. Next, a placement position for the vascular graft 10_{d} in the aortic arch 110 can be determined using any one or combination of known imaging techniques, such as fluoroscopy, angiography, ultrasonography, CT, helical CT, CT angiogram, MRI, and/or MR angiography.

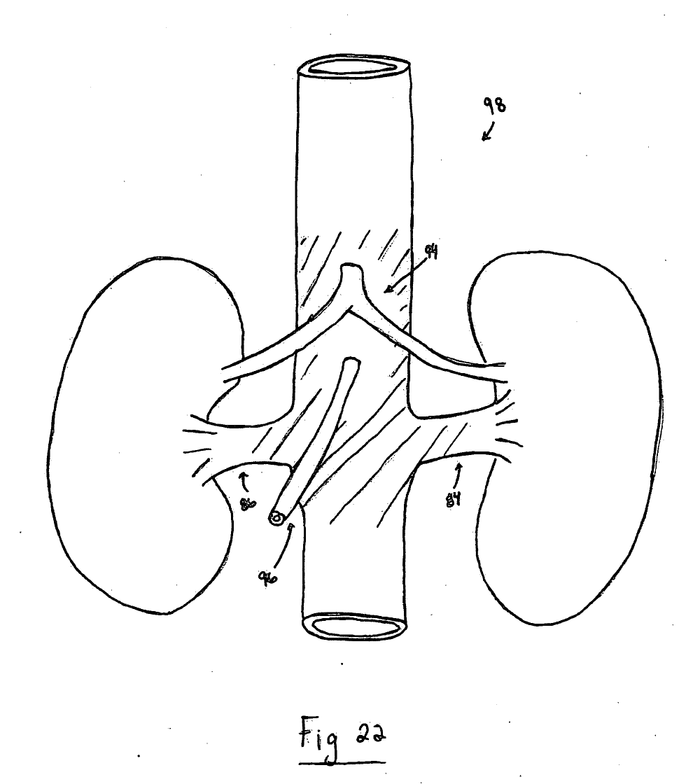

Prior to implanting the vascular graft 10_{d}, the vascular graft can be loaded into a delivery sheath 100 (FIGS. 18 and 19) to facilitate delivery of the vascular graft. The delivery sheath 100 can maintain the vascular graft 10_{d} in a sterile environment while also keeping the vascular graft in a compressed configuration until implantation. As shown in FIGS. 18 and 19, the delivery sheath 100 can be constructed in an identical or similar manner as described above. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{d} to a desired location.

After loading the main body portion 12 and the second main body portion 64_{d} into the first and second envelope members 102 and 104, respectively, the second envelope member 104 containing the second main body portion can be delivered to the proximal portion 114 of the descending aorta 112 (FIG. 18). The expandable support member 44 of the second main body portion 64_{d} can include a radio-opaque marker (not shown) to facilitate implantation of the second main body portion. The string 108 or line of the second envelope member 104 can then be manipulated (e.g., pulled) so that the second envelope member is released from the second main body portion 64_{d}. As the second envelope member 104 is withdrawn from the second main body portion 64_{d}, the expandable support member 44 can obtain an expanded configuration and thereby cause the second main body portion to expand into the proximal portion 114 of the descending aorta 112 (FIG. 19).

After the second main body portion 64_{d} is securely positioned in the proximal portion 114 of the descending aorta 112, the first envelope member 102 can be delivered to the aortic arch 110 so that the second end 28 of the main body portion 12 is positioned substantially adjacent the first end 66_{d} of the second main body portion. With the second main body portion 64_{d} securely positioned in the proximal portion 114 of the descending aorta 112, the second main body portion 64_{d} can serve as a platform to facilitate attachment of the main body portion 12 to the first end 66_{d} of the second main body portion.

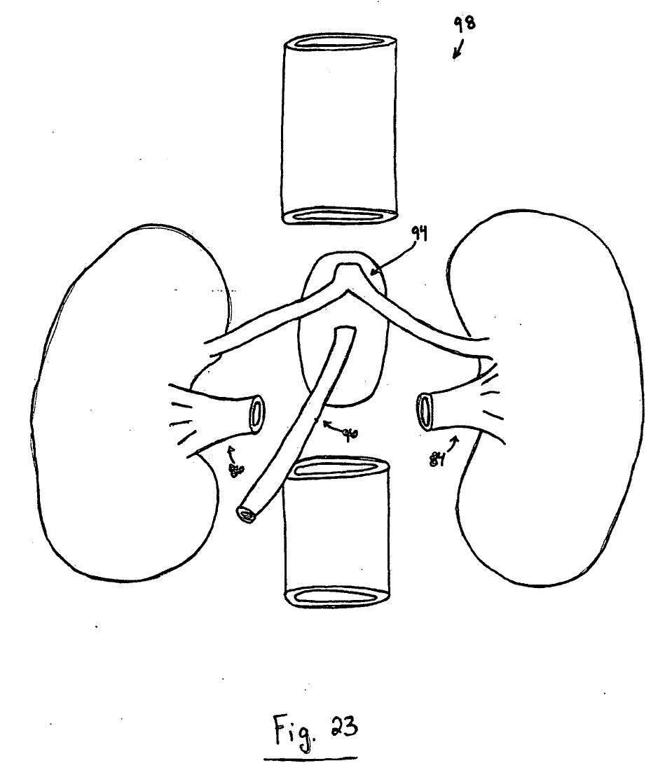

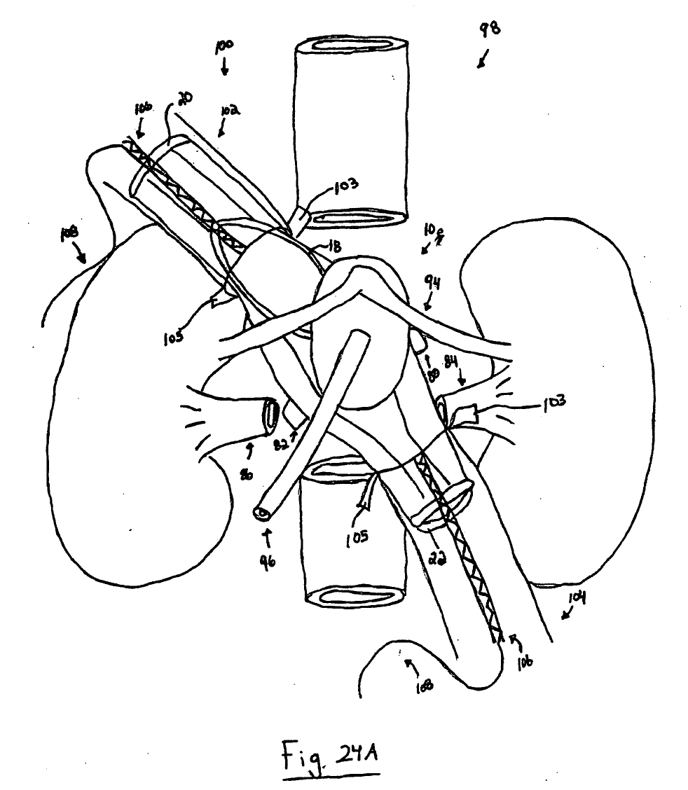

Once the main body portion 12 and the second main body portion 64_{d} of the vascular graft 10_{d} are positioned adjacent one another, the first end 66_{d} of the second main body portion can be anastomosed with the third sewing ring 22. After the main body portion 12 and second main body portion 64_{d} are securely attached to one another, the first envelope member 102 can be withdrawn so that the main body portion expands into the aortic arch 110 (FIG. 20). As shown in FIG. 21, the first, second and third branch members 52, 54, and 56 of the multi-lumen branch graft 16 can then be respectively anastomosed with the brachiocephalic trunk artery 58, the left common carotid artery 60, and the left subclavian artery 62 using sutures, for example. Additionally, the fourth sewing ring 78 can be secured in the descending aorta 112 using sutures.

Although not shown in FIG. 21, the first sewing ring 18 of the vascular graft 10_{d} can be stitched to the aortic arch 110. It will be appreciated that the second sewing ring 20 can be secured to the ascending aorta (not shown in detail), the aortic root (not shown), or can be used to secure a mechanical or biological bioprosthetic valve (not shown) in a native cardiac valve (not shown). After the vascular graft 10_{d} is secured in the aortic arch 110, the incision in the aortic arch can be closed and the vessels surrounding the aortic arch undamped so that blood may flow normally through the vascular graft.

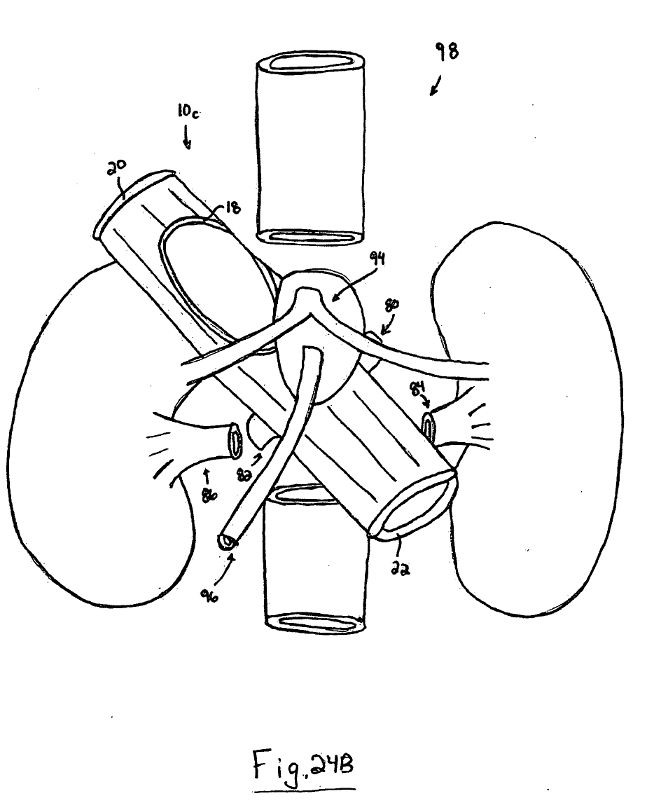

In another embodiment of the present invention, a method is provided for repairing a portion of an abdominal aorta 98 (FIG. 22) in a subject. The portion of the abdominal aorta 98 to be repaired can include a portion of the abdominal aorta affected by an aneurysm. To repair a AAA, for example, an open-chest surgical procedure using the vascular graft 10ₑ shown in FIG. 6A can be employed. Although implantation of the vascular graft 10ₑ is described below using an open-abdominal surgical approach, it will be appreciated that other methods for implanting the vascular graft, such as a percutaneous or minimally invasive surgical technique may also be used.

To repair the AAA, a vascular graft 10ₑ as illustrated in FIG. 6A can first be provided. Next, a placement position for the vascular graft 10_{c} in the abdominal aorta 98 can be determined using one or a combination of known imaging techniques such as ultrasonography, CT, helical CT, CT angiogram, MRI, and/or MR angiography. After identifying the placement position, the subject can be prepared for surgery.

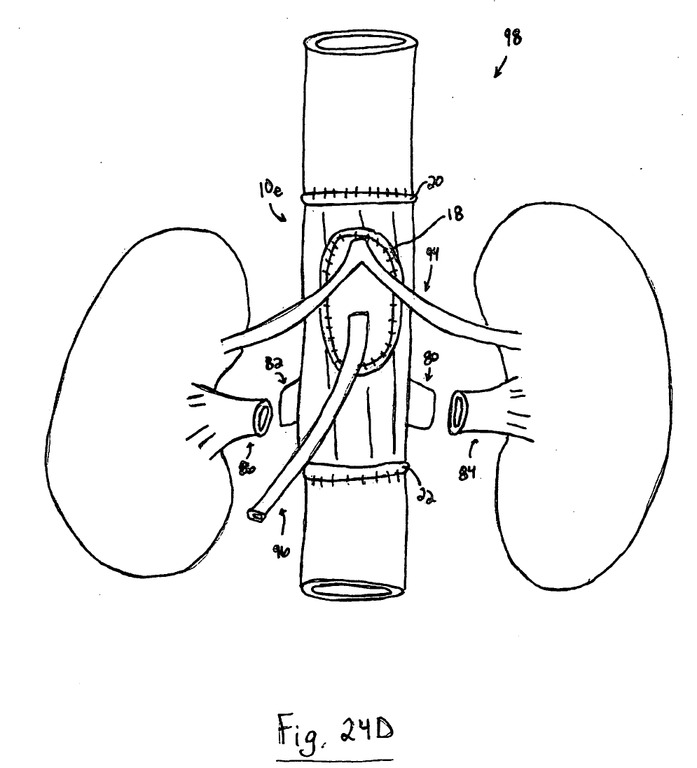

Prior to implantation of the vascular graft 10ₑ, the vascular graft can be loaded into a delivery sheath 100 (FIG. 24A) to facilitate delivery of the vascular graft. The delivery sheath 100 maintains the vascular graft 10ₑ in a sterile environment while also keeping the vascular graft in a compressed configuration prior to implantation. Although not shown in detail, the delivery sheath 100 comprises first and second envelope members 102 and 104 capable of containing respective portions of the vascular graft 10ₑ in a compressed configuration. Each of the first and second envelope members 102 and 104 includes a release mechanism 106 (or any other suitable alternative) for selectively releasing the vascular graft 10ₑ from the delivery sheath 100.

The mechanism 106 can include at least one string 108 or line which, when pulled or retracted, causes the mechanism to separate each of the first and second envelope members 102 and 104 and thereby release the respective portions of the vascular graft 10ₑ from the envelope members. The delivery sheath 100 can be made of a transparent, biocompatible material (e.g., a plastic polymer) to facilitate visualization of the vascular graft 10ₑ during implantation. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{c} to a desired location.

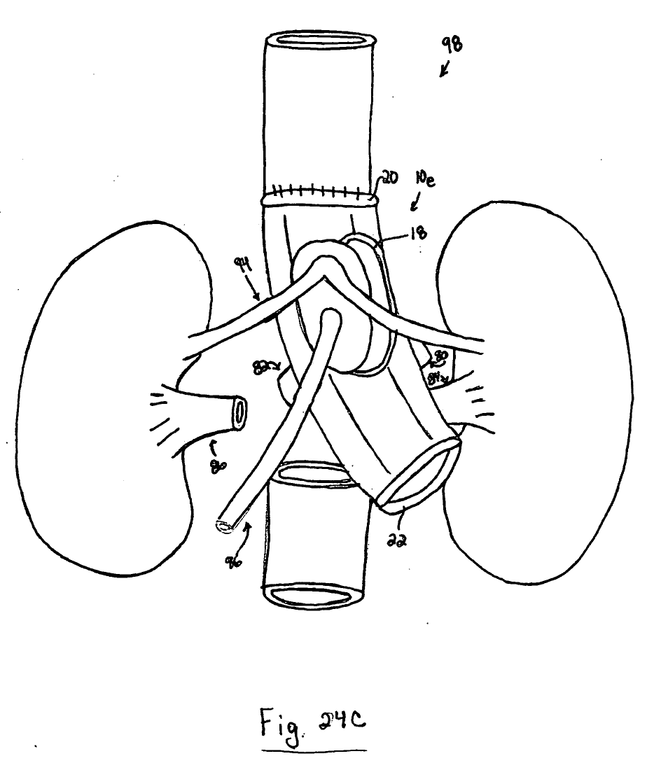

After loading the vascular graft 10ₑ into the delivery sheath 100, an incision (not shown) can then be made over the skin of the subject and through the muscle (not shown) overlying the abdominal aorta 98. The abdominal tissue (not shown) surrounding the abdominal aorta 98 can then be manipulated to clearly expose the AAA. Next, the vessels superior and inferior to the AAA can be tied off or clamped (not shown) to temporarily stop blood flow through the abdominal aorta 98. For example, portions of the abdominal aorta 98 both superior and inferior to the AAA can be clamped. After clamping the vessels surrounding the AAA, the diseased portion (FIG. 22; shaded region) of the abdominal aorta 98 can be resected as shown in FIG. 23.

Next, the delivery sheath 100 containing the vascular graft 10ₑ can be positioned over the abdominal aorta 98 as shown in FIG. 24A. More particularly, the first end 24 of the vascular graft 10ₑ can be positioned adjacent the abdominal aorta 98 superior to the left and right renal arteries 84 and 86, and the second end 28 of the vascular graft positioned adjacent the abdominal aorta inferior to the renal arteries. Next, the string 108 or line of each of the first and second envelope members 102 and 104 can be manipulated (e.g., pulled) so that the first and second envelope members begin to release the vascular graft 10ₑ. As the first and second envelope members 102 and 104 release the vascular graft 10ₑ, the main body portion 12ₑ can begin to expand into the AAA (FIG. 24B). Upon completely removing the delivery sheath 100 from the vascular graft 10e, the second sewing ring 20 can be anastomosed with the superior portion of the abdominal aorta 98 (FIG. 24C). Next, the third sewing ring 22 can be anastomosed with the inferior portion of the abdominal aorta 98 (FIG. 24D).

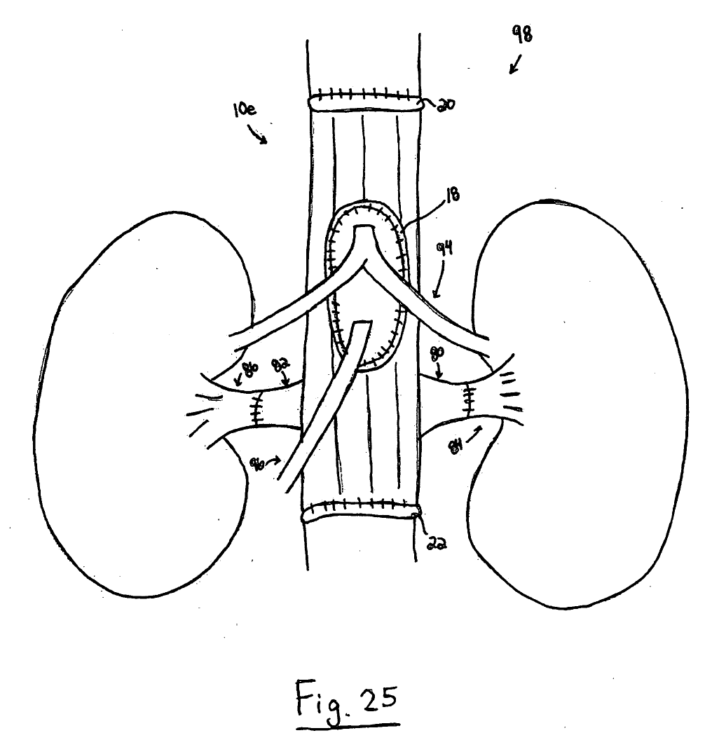

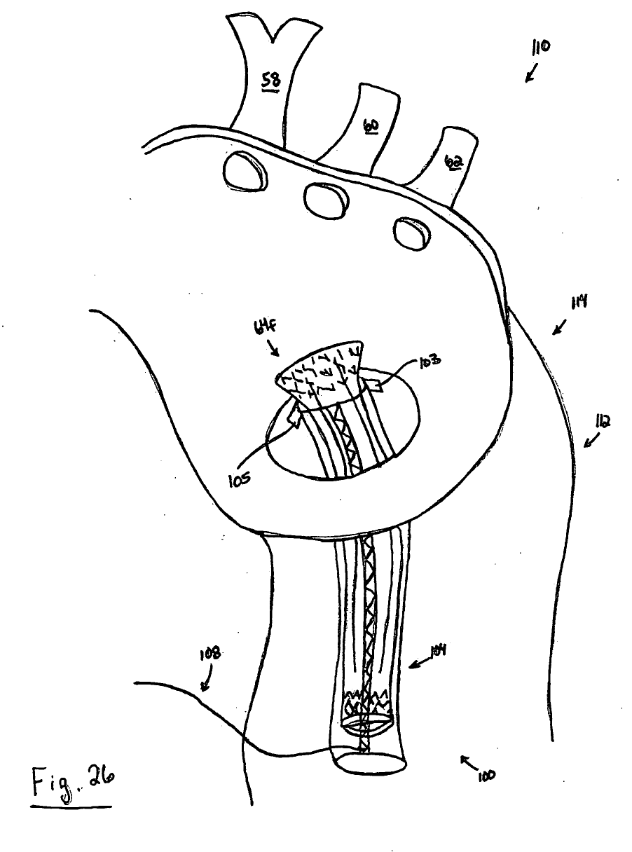

After stitching the second and third sewing rings 20 and 22 to the abdominal aorta 98 as shown in FIG. 24D, the left and right renal arteries 84 and 86 can be respectively anastomosed with the first and second arm members 80 and 82 of the vascular graft 10ₑ. Additionally, the portion of the aortic wall which includes the celiac trunk 94 and the superior mesenteric artery 96 can be anastomosed or stitched to the first sewing ring 18. After the vascular graft 10ₑ is securely positioned in the abdominal aorta 98 (FIG. 25), the clamps can be removed and normal blood flow can resume through the vascular graft. To complete the surgery, the abdominal tissue can be returned to its place over the abdominal aorta 98 and the incision closed with sutures.

In another embodiment of the present invention, a method is provided for repairing a thoracic aortic aneurysm, such as an aneurysm of the aortic arch 110 (FIG. 26) in a subject using a multi-stage approach. The multi-stage approach can be performed via an open-chest elephant trunk procedure to repair an aortic arch aneurysm. It will be appreciated, however, that other surgical approaches, such as a minimally invasive or percutaneous approach may also be used.

To repair the aortic arch 110 using a multi-stage approach (i.e., an elephant trunk procedure), a vascular graft 10_{f}, such as the one illustrated in FIG. 7B can first be provided. Next, a placement position for the vascular graft 10_{f} in the aortic arch 110 can be determined using any one or combination of known imaging techniques, such as ultrasonography, CT, helical CT, CT angiogram, MRI, and/or MR angiography.

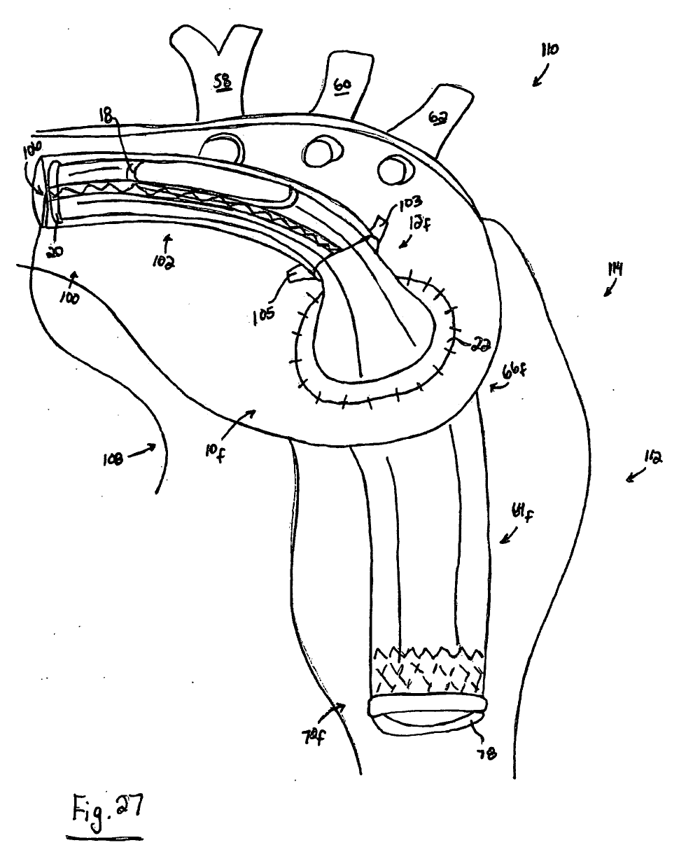

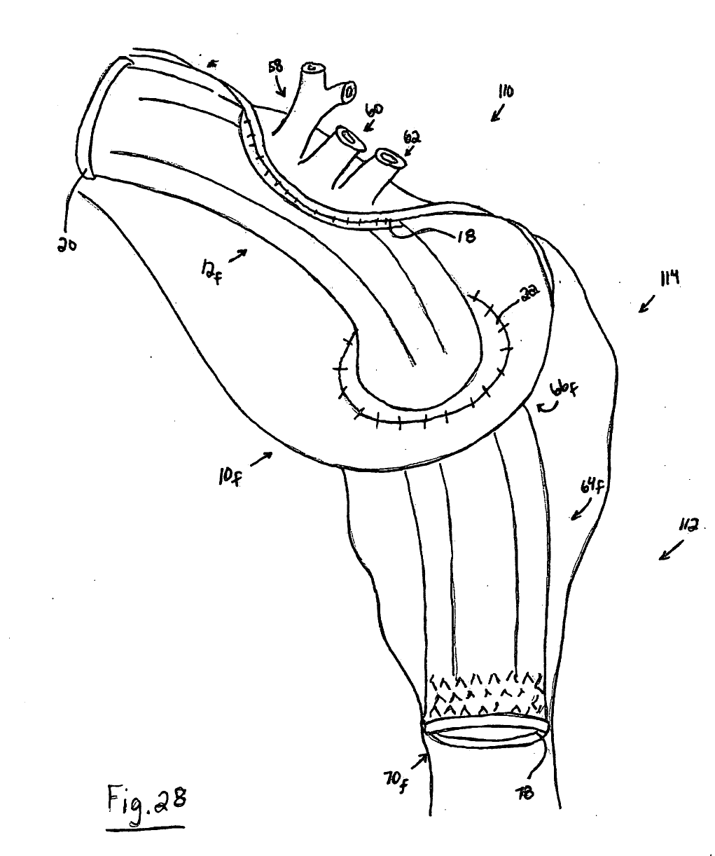

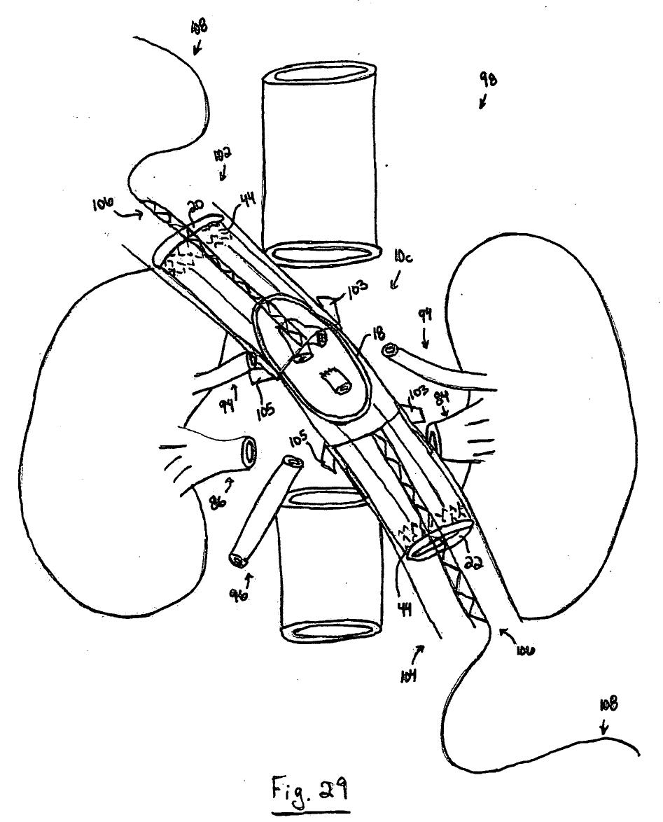

Prior to implanting the vascular graft 10_{f}, the vascular graft can be loaded into a delivery sheath 100 (FIGS. 26 and 27) to facilitate delivery of the vascular graft. The delivery sheath 100 can maintain the vascular graft 10_{f} in a sterile environment while also keeping the vascular graft in a compressed configuration until implantation. As shown in FIGS. 26 and 27, the delivery sheath 100 can be constructed in an identical or similar manner as described above. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{f} to a desired location.

After loading the main body portion 12_{f} and the second main body portion 64_{f} into the first and second envelope members 102 and 104, respectively, the vascular graft 10_{f} can be implanted using an open-chest procedure. As shown in FIG. 26, the second envelope member 104 containing the second main body portion 64_{f} can be delivered to the proximal portion 114 of the descending aorta 112. Each of the expandable support members 44 of the second main body portion 64_{f} can include a radio-opaque marker (not shown) to facilitate implantation of the second main body portion. The string 108 or line of the second envelope member 104 can then be manipulated (e.g., pulled) so that the second envelope member is released from the second main body portion 64_{f}. As the second envelope member 104 is withdrawn from the second main body portion 64_{f}, the expandable support members 44 can obtain an expanded configuration and thereby cause the second main body portion to expand into the proximal portion 114 of the descending aorta 112 (FIG. 27).

After the second main body portion 64_{f} is securely positioned in the proximal portion 114 of the descending aorta 112, the first envelope member 102 can be delivered to the aortic arch 110 so that the second end 28 of the main body portion 12 is positioned substantially adjacent the first end 66_{f} of the second main body portion. With the second main body portion 64_{f} securely positioned in the proximal portion 114 of the descending aorta 112, the second main body portion 64_{f} can serve as a platform to facilitate attachment of the main body portion 12 to the first end 66_{f} of the second main body portion.

Once the main body portion 12_{f} and the second main body portion 64_{f} of the vascular graft 10_{f} are positioned adjacent one another, the first end 66_{f} of the second main body portion can be anastomosed with the third sewing ring 22. After the main body portion 12_{f} and second main body portion 64_{f} are securely attached to one another, the first envelope member 102 can be withdrawn so that the main body portion expands into the aortic arch 110 (FIG. 27). As shown in FIG. 28, the first sewing ring 18 can then be respectively anastomosed with a portion of the aortic arch 110 including the brachiocephalic trunk artery 58, the left common carotid artery 60, and the left subclavian artery 62 using sutures, for example. Additionally, the fourth sewing ring 78 of the vascular graft 10_{f} can be stitched to the descending aorta 112 using sutures, for example, or to another vascular graft as part of a second-stage elephant trunk procedure.

Although not shown in FIG. 28, the second sewing ring 20 of the vascular graft 10_{f} can be stitched to the aortic arch 110. After the vascular graft 10_{f} is secured in the aortic arch 110, the incision in the aortic arch can be closed and the vessels surrounding the aortic arch undamped so that blood may flow normally through the vascular graft.

In another embodiment of the present invention, a method is provided for repairing a portion of an abdominal aorta 98 (FIG. 10) in a subject. To repair a AAA, a vascular graft 10_{c} similar to the vascular graft illustrated in FIGS. 4A and 48 can first be provided. The vascular graft 10_{c} in FIGS. 29-31 is identical to the vascular graft in FIGS. 4A and 4B, except that the first and second ends 24 and 28 of the vascular graft in FIGS. 29-31 each include an expandable support member 44.

A placement position for the vascular graft 10_{c} in the abdominal aorta 98 can be determined using one or a combination of known imaging techniques, such as ultrasonography, fluoroscopy, angiography, CT, helical CT, CT angiogram, MRI, and/or MR angiography. After identifying the placement position, the subject may be prepared for surgery. Although implantation of the vascular graft 10_{c} is described below using an open-abdominal surgical approach, it will be appreciated that other methods for implanting the vascular graft, such as a percutaneous or minimally invasive surgical technique may also be used.

Prior to implantation of the vascular graft 10_{c}, the vascular graft can be loaded into a delivery sheath 100 (FIG. 29) to facilitate delivery of the vascular graft. The delivery sheath 100 maintains the vascular graft 10_{c} in a sterile environment while also keeping the vascular graft in a compressed configuration prior to implantation. Although not shown in detail, the delivery sheath 100 comprises first and second envelope members 102 and 104 capable of containing respective portions of the vascular graft 10_{c} in a compressed configuration. Each of the first and second envelope members 102 and 104 includes a release mechanism 106 (or any other suitable alternative) for selectively releasing the vascular graft 10_{c} from the delivery sheath 100.

The mechanism 106 can include at least one string 108 or line which, when pulled or retracted, causes the mechanism to separate each of the first and second envelope members 102 and 104 and thereby release the respective portions of the vascular graft 10_{c} from the envelope members. The delivery sheath 100 can be made of a transparent, biocompatible material (e.g., a plastic polymer) to facilitate visualization of the vascular graft 10_{c} during implantation. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{c} to a desired location.

After loading the vascular graft 10_{c} into the delivery sheath 100, an incision (not shown) can be made over the skin of the subject and through the muscle (not shown) overlying the abdominal aorta 98. The abdominal tissue (not shown) surrounding the abdominal aorta 98 can then be manipulated to clearly expose the AAA. Next, the vessels superior and inferior to the AAA can be tied off or clamped (not shown) to temporarily stop blood flow through the abdominal aorta 98. For example, portions of the abdominal aorta 98 both superior and inferior to the AAA can be clamped. Additionally, portions of the left and right renal arteries 84 and 86, as well as the celiac trunk 94 and superior mesenteric artery 96 may be clamped to temporarily prevent blood flow through the AAA. After clamping the vessels surrounding AAA, the diseased portion of the abdominal aorta 98 can be resected as described above.

Next, the delivery sheath 100 containing the vascular graft 10_{c} can be positioned over the abdominal aorta 98 as shown in FIG. 29. More particularly, the first end 24 of the vascular graft 10_{b} can be positioned adjacent the abdominal aorta 98 superior to the left and right renal arteries 84 and 86, and the second end 28 of the vascular graft can be positioned adjacent the abdominal aorta inferior to the renal arteries.

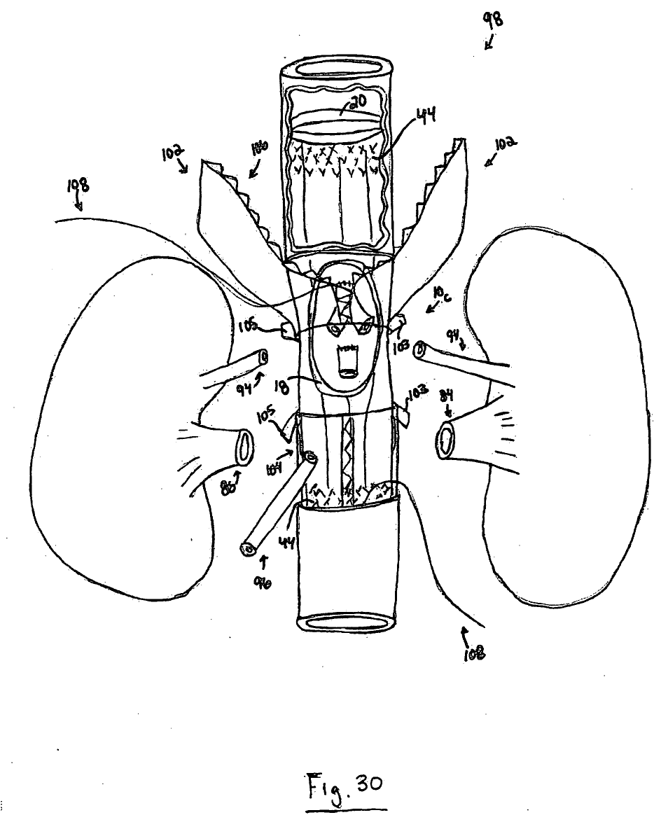

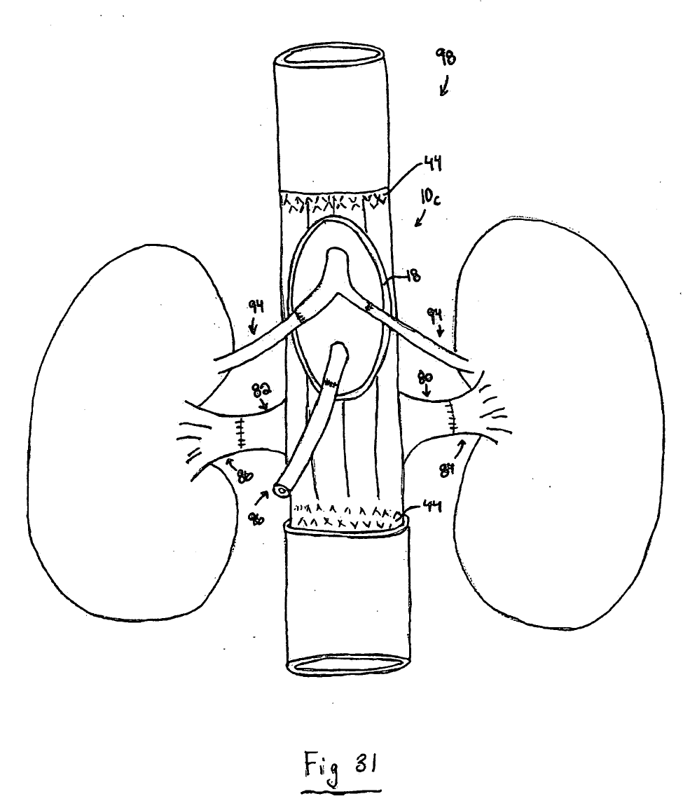

As shown in FIG. 30, the first and second ends 24 and 28 of the vascular graft 10_{c} (in a compressed configuration) can then be respectively inserted into the superior and inferior portions of the abdominal aorta 98. Then, the string 108 or line of the first envelope member 102 can be manipulated (e.g., pulled) so that the first envelope member is peeled away and progressively releases the vascular graft 10_{c} into the abdominal aorta 98. As the first envelope member 102 releases the vascular graft 10_{c}, the vascular graft expands via the expandable support member 44 into the superior portion of the abdominal aorta 98 and securely engages the lumen of the abdominal aorta (FIG. 30).

Next, the release mechanism 106 of the second envelope member 104 can be manipulated (i.e., pulled) so that the second envelope member is peeled away and the vascular graft 10_{c} expands via the expandable support member 44 to engage the lumen of the inferior abdominal aorta 98. It should be appreciated that the order in which the first and second envelope members 102 and 104 are peeled away from the vascular graft 10_{c} is not limited to the order illustrated above. For example, the second envelope member 104 can be peeled away from the vascular graft 10_{c} before the first envelope member 102 or, alternatively, the first and second envelope members may be simultaneously peeled away from the vascular graft. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{c} to the desired location.

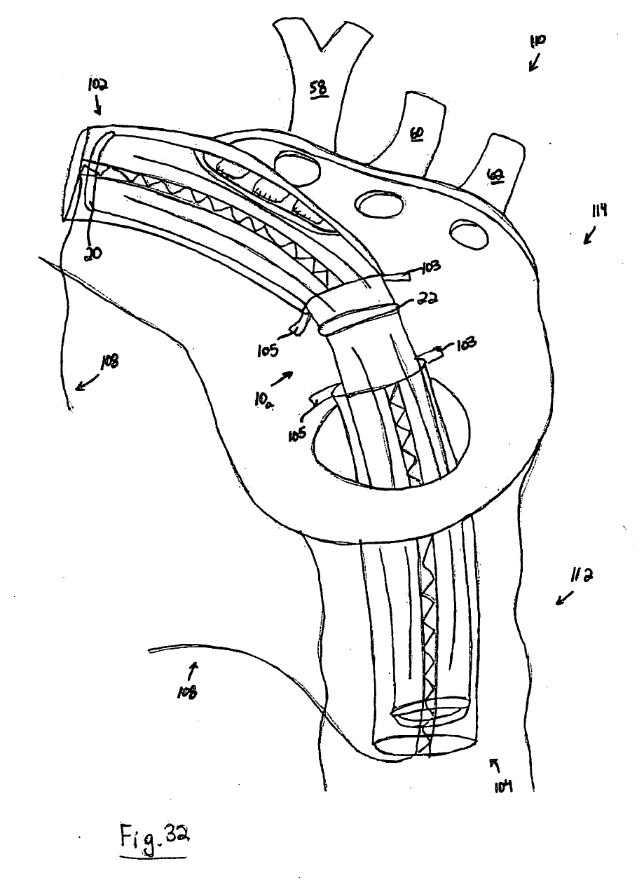

Upon completely removing the delivery sheath 100 from the vascular graft 10_{c}, the left and right renal arteries 84 and 86 can be respectively anastomosed with the first and second arm members 80 and 82 of the vascular graft 10_{c}. Next, the celiac trunk 94 and the superior mesenteric artery 96 can be respectively anastomosed with the first and second branch members 52_{c} and 54_{c}. After the vascular graft 10_{c} is securely positioned in the abdominal aorta 98 (FIG. 31), the clamps can be removed and normal blood flow can resume through the vascular graft. To complete the surgery, the abdominal tissue may be returned to its place over the abdominal aorta 98 and the incision closed with sutures.

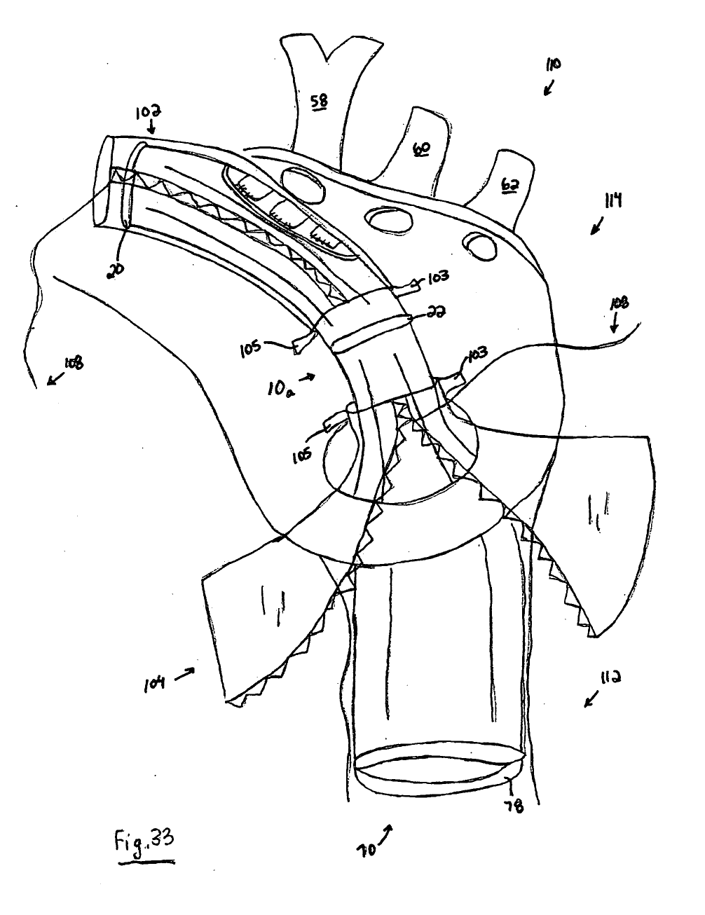

In another embodiment of the present invention, a method is provided for repairing a thoracic aortic aneurysm, such as an aneurysm of the aortic arch 110 (FIG. 32) in a subject. To repair an aortic arch aneurysm, an open-chest elephant trunk procedure using the vascular graft 10_{b} shown in FIGS. 3A and 3B can be employed. Although implantation of the vascular graft 10_{b} is described below using an open surgical approach, it will be appreciated that other methods for implanting the vascular graft, such as a percutaneous or minimally invasive surgical technique may also be used.

To repair the aortic arch aneurysm, the vascular graft 10_{b} illustrated in FIGS. 3A and 3B can first be provided. Next, a placement position for the vascular graft 10_{b} in the aortic arch aneurysm can be determined using a known imaging technique such as fluoroscopy, angiography, ultrasonography, CT, helical CT, CT angiogram, MRI, and/or MR angiography.

Prior to implanting the vascular graft 10_{b}, the vascular graft can be loaded into a delivery sheath 100 (FIG. 32) to facilitate delivery of the vascular graft. The delivery sheath 100 can maintain the vascular graft 10_{b} in a sterile environment while also keeping the vascular graft in a compressed configuration prior to implantation. As shown in FIG. 32, the delivery sheath 100 can be constructed in an identical or similar manner as described above. It will be appreciated that the release mechanism 106 can also include first and second tab members 103 and 105. The first and second tab members 103 and 105 can be manipulated by, for example, tactile means to progressively peel away the delivery sheath 100 and thereby deliver the vascular graft 10_{b} to a desired location.

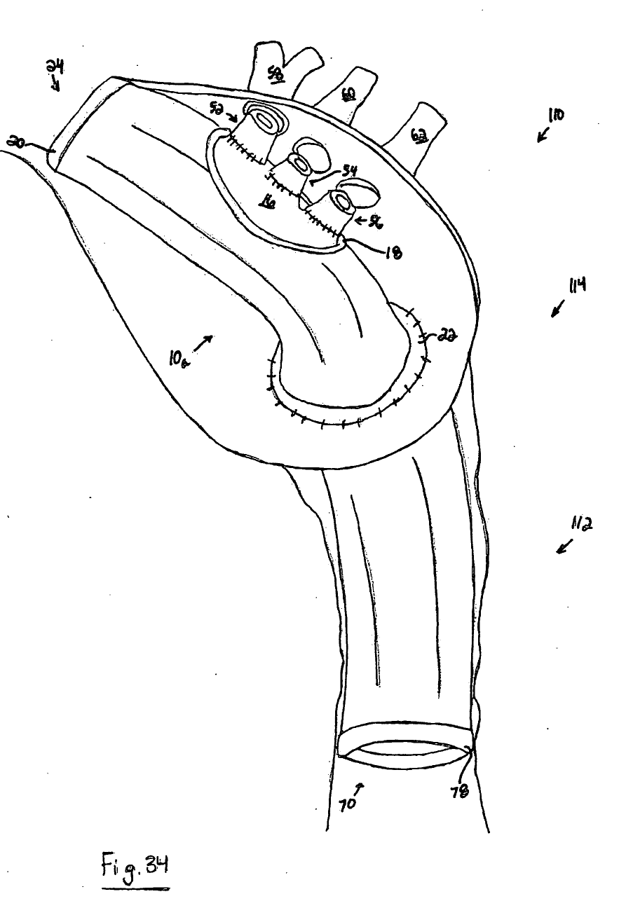

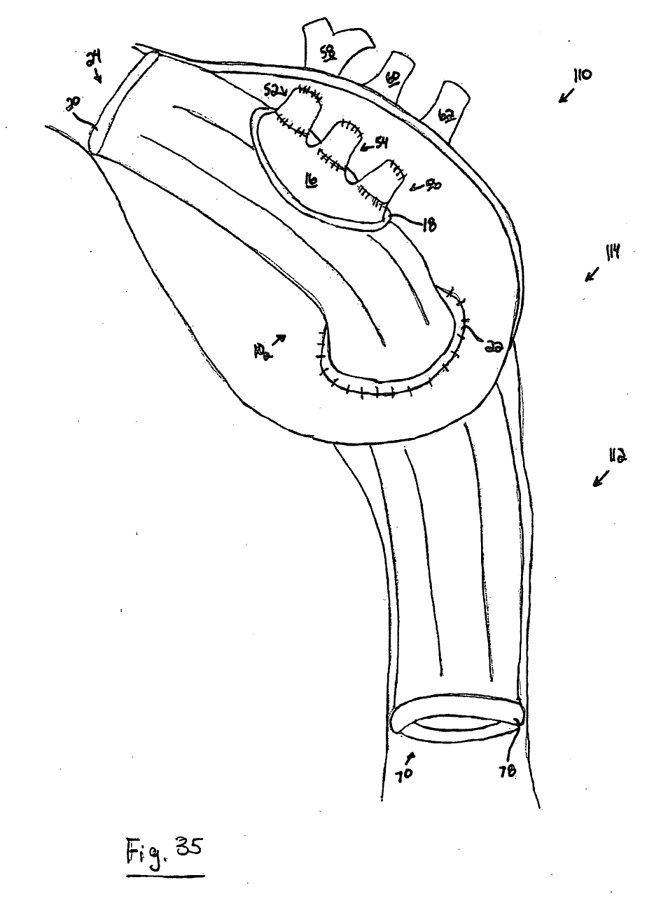

After loading the vascular graft 10_{b} into the delivery sheath 100, the delivery sheath 100 containing the vascular graft 10_{b} can be inserted into the aortic arch 110 via an incision. As shown in FIG. 32, the second main body portion 64 of the vascular graft 10_{b} can be positioned in the descending aorta 112. After positioning the second main body portion 64 in the descending aorta 112, the string 108 or line of the second envelope member 104 can be manipulated (e.g., pulled) so that the second envelope member releases the second main body portion and the second main body portion expands into the descending aorta (FIG. 33). The third sewing ring 22 can then be stitched to the proximal portion 114 of the descending aorta 112 (FIG. 34).

After securely attaching the third sewing ring 22 to the proximal portion 114 of the descending aorta 112, the string 108 or line of the first envelope member 102 can be manipulated (e.g., pulled) so that the first envelope member releases the main body portion 12 and the main body portion expands into the aortic arch 110 (FIG. 34). As shown in FIG. 35, the first, second and third branch members 52, 54, and 56 of the multi-lumen branch graft 16 can then be respectively anastomosed with the brachiocephalic trunk artery 58, the left common carotid artery 60, and the left subclavian artery 62. It will be appreciated that the second sewing ring 20 can also be secured to the ascending aorta (not shown in detail), the aortic root (not shown), or used to secure a mechanical or biological bioprosthetic valve (not shown) in a native cardiac valve (not shown). After the vascular graft 10_{b} is secured in place of the aortic arch aneurysm, the incision in the aortic arch 110 can be closed and the vessels surrounding the vascular graft undamped so that blood can flow normally through the vascular graft.

From the above description of the invention, those skilled in the art will perceive improvements, changes and modifications. For example, it will be appreciated that the order of steps described above for implanting the present invention are intended to be illustrative only and are not intended to limit the present inventive method to the order of steps described herein. Such improvements, changes, and modifications are within the skill of the art and are intended to be covered by the appended claims.

Having described the invention,

1

claim:

1. A vascular graft comprising:
an elongated main body portion including a first end defining a first opening, a second end defining a second opening, and an aperture defined by a perimeter;
a first sewing ring securely attached to said perimeter of said aperture;
a second sewing ring securely attached to said first end of said main body portion adjacent said first opening; and
a third sewing ring securely attached to said second end of said main body portion adjacent said second opening.

2. The vascular graft of claim 1, wherein said main body portion includes a lumen extending between said first and second ends and being defined by an inner surface, said main body portion further comprising at least one expandable support member operably secured to said inner surface of said lumen.

3. The vascular graft of claim 2, wherein first and second expandable support members are operably secured at said first and second ends of said main body portion.

4. The vascular graft of claim 1 further comprising a multi-lumen branch graft including a plurality of branch members and an attachment portion adapted for anastomosis with said first sewing ring, each of said branch members having first and second ends, each of said first ends of said branch members being securely attached to said attachment portion.

5. The vascular graft of claim 4, wherein said multi-lumen branch graft comprises a first branch member configured to accommodate a portion of a brachiocephalic trunk artery, a second branch member configured to accommodate a portion of a left common carotid artery, and a third branch member configured to accommodate a portion of a left subclavian artery.

6. The vascular graft of claim 4, wherein said multi-lumen branch graft includes at least one branch member configured to accommodate a portion of at least one blood vessel selected from the group consisting of a celiac trunk artery, a superior mesenteric artery, an inferior mesenteric artery, an inferior phrenic artery, a middle suprarenal artery, a gonadal artery, a lumbar artery, and a middle sacral artery.

7. The vascular graft of claim 4, wherein said multi-lumen branch graft comprises a first branch member configured to accommodate a portion of a celiac trunk artery and a second branch member configured to accommodate a portion of a superior mesenteric artery.

8. The vascular graft of claim 1 further comprising oppositely disposed first and second arm members securely attached to said main body portion, said first and second arm members respectively configured to accommodate a portion of a left renal artery and a portion of a right renal artery.

9. The vascular graft of claim 4 further comprising a second main body portion having a first end defining a first opening and a second end defining a second opening, said first end being securely attached to said third sewing ring of said main body portion, said second end of said second main body portion having a fourth sewing ring securely attached thereto and being adjacent said second opening.

10. The vascular graft of claim 9, wherein said second main body portion includes a lumen extending between said first and second ends and being defined by an inner surface, said second main body portion further comprising at least one expandable support member operably secured to said inner surface of said lumen.

11. The vascular graft of claim 1, wherein said first sewing ring is adapted for anastomosis with a portion of an aortic arch including a brachiocephalic trunk artery, a left common carotid artery, and a left subclavian artery.

12. The vascular graft of claim 1, wherein said first sewing ring is adapted for anastomosis with a portion of an abdominal aorta including at least one blood vessel selected from the group consisting of a celiac trunk artery, a superior mesenteric artery, an inferior mesenteric artery, an inferior phrenic artery, a middle suprarenal artery, a gonadal artery, a lumbar artery, and a middle sacral artery.

13. The vascular graft of claim 1 further comprising a delivery sheath for delivering said vascular graft to a portion of a blood vessel to be repaired, said delivery sheath comprising first and second envelope members for containing respective portions of said vascular graft, each of said first and second envelope members including a release mechanism for selectively releasing said vascular graft from said delivery sheath.

14. The apparatus of claim 13, wherein said release mechanism comprises a zipper and at least one line operably connected thereto for actuating said zipper.

15. A vascular graft comprising:
a Y-shaped tubular main body portion having first and second ends, said first end defining a first opening and having a first sewing ring securely attached thereto, said second end including first and second leg members fluidly connected with said main body portion, each of said first and second leg members including an end defined by an opening and having second and third sewing rings respectively attached thereto, said main body portion including a fourth sewing ring securely attached thereto.

16. The vascular graft of claim 15, wherein each of said ends of said vascular graft includes at least one expandable support member securely disposed therein.

17. The vascular graft of claim 15, wherein fifth and sixth sewing rings are securely attached to said first and second leg members.

18. The vascular graft of claim 17, wherein said fifth sewing ring is intermediate said second and fourth sewing rings and said sixth sewing ring is intermediate said third and fourth sewing rings.

19. A method for repairing at least a portion of a blood vessel, said method comprising the steps of:
providing an apparatus comprising a vascular graft at least partially encapsulated within a delivery sheath, the vascular graft including an elongated main body portion, a first sewing ring, a second sewing ring, and a third sewing ring, the first sewing ring being securely attached to an aperture of the main body portion, the second sewing ring being securely attached to a first end of the main body portion adjacent a first opening, the third sewing ring being securely attached to a second end of the main body portion adjacent a second opening, the delivery sheath comprising first and second envelope members, each of the first and second envelope members including a release mechanism;
determining a placement position for the vascular graft at the portion of the blood vessel to be repaired;
delivering the vascular graft to the portion of the blood vessel to be repaired;
manipulating the delivery sheath so that the vascular graft expands into the portion of the blood vessel to be repaired; and
securing the vascular graft to the portion of the blood vessel to be repaired.

20. The method of claim 19 further comprising the steps of:
operating the release mechanism of the delivery sheath so that the first and second envelope members release the vascular graft at an aortic arch aneurysm;
attaching the second sewing ring to a portion of the ascending aorta;
attaching the third sewing ring to a portion of the descending aorta; and
attaching the first sewing ring to a portion of the aortic arch, the portion of the aortic arch comprising a portion of the brachiocephalic trunk artery, a portion of the left common carotid artery, and a portion of the left subclavian artery.

21. The method of claim 19 further comprising the steps of:
operating the release mechanism of the delivery sheath so that the first and second envelope members release the vascular graft at an abdominal aortic aneurysm;
attaching the second sewing ring to a first portion of the abdominal aorta;
attaching the third sewing ring to a second portion of the abdominal aorta; and
attaching the first sewing ring to a third portion of the abdominal aorta, the third portion of the abdominal aorta comprising a portion of the celiac trunk artery and a portion of the superior mesenteric artery.

22. The method of claim 19, wherein said step of providing a vascular graft further comprises providing a multi-lumen branch graft including first, second, and third branch members and an attachment portion adapted for anastomosis with the first sewing ring, each of the branch members having first and second ends, the first end of each of the branch members being securely attached to the attachment portion.

23. The method of claim 22 further comprising the steps of:
operating the release mechanism of the delivery sheath so that the first and second envelope members release the vascular graft at an aortic arch aneurysm;
attaching the second sewing ring to a portion of the ascending aorta;
attaching the third sewing ring to a portion of the descending aorta; and
attaching the first, second, and third branch members to a portion of the brachiocephalic trunk artery, a portion of the left common carotid artery, and a portion of the left subclavian artery, respectively.

24. The method of claim 19, wherein said step of providing a vascular graft further comprises providing a multi-lumen branch graft including first and second branch members and an attachment portion adapted for anastomosis with the first sewing ring, each of the branch members having first and second ends, the first end of each of the branch members being securely attached to the attachment portion

25. The method of claim 24 further comprising the steps of:
operating the release mechanism of the delivery sheath so that the first and second envelope members release the vascular graft at abdominal aortic aneurysm;
attaching the second sewing ring to a first portion of the abdominal aorta;
attaching the third sewing ring to a second portion of the abdominal aorta;
attaching the first branch member to a portion of the celiac trunk artery; and
attaching the second branch member to a portion of the superior mesenteric artery.

26. The method of claim 24 further comprising the step of providing oppositely disposed first and second arm members, the first and second arm members being securely attached to the main body portion and respectively configured to accommodate a portion of the left renal artery and a portion of the right renal artery.

27. The method of claim 26 further comprising the steps of:
operating the release mechanism of the delivery sheath so that the first and second envelope members release the vascular graft at an abdominal aortic aneurysm;
attaching the second sewing ring to a first portion of the abdominal aorta;
attaching the third sewing ring to a second portion of the abdominal aorta;
attaching the first branch member to a portion of the celiac trunk artery;
attaching the second branch member to a portion of the superior mesenteric artery;
attaching the first arm member to a portion of the left renal artery; and
attaching the second arm member to a portion of the right renal artery.

28. The method of claim 19, wherein said step of providing a vascular graft further comprises the steps of:
providing a multi-lumen branch graft including first, second, and third branch members and an attachment portion adapted for anastomosis with the first sewing ring, each of the branch members having first and second ends, the first end of each of the branch members being securely attached to the attachment portion; and
providing a second main body portion having a first end defining a first opening and a second end defining a second opening, the first end being securely attached to the third sewing ring of the main body portion, the second end of the second main body portion having a fourth sewing ring securely attached thereto and being adjacent the second opening.

29. The method of claim 28 further comprising the steps of:
operating the release mechanism of the delivery sheath so that the first and second envelope members release the vascular graft at an aortic arch aneurysm;
attaching the third sewing ring to a proximal portion of the descending aorta;
attaching the second sewing ring to a portion of the aortic arch;
attaching the fourth sewing ring to a portion of the descending aorta; and
attaching the first, second, and third branch members to a portion of the brachiocephalic trunk artery, a portion of the left common carotid artery, and a portion of the left subclavian artery, respectively.
